(19) United States
(12) Patent Application Publication
(10) Pub. No.: US 2021/0253537 A1
(43) Pub. Date: Aug. 19, 2021

(54) HYDROGENATION PROCESS

(71) Applicant: JOHNSON MATTHEY PUBLIC LIMITED COMPANY, London (GB)

(72) Inventors: Paul GORDON, Thornaby (GB); Steve POLLINGTON, Billingham, Cleveland (GB); John SWINNEY, Thornaby (GB); Michael David HAMLIN, Devens, MA (US); Steven James COLLIER, Devens, MA (US)

(21) Appl. No.: 16/973,678

(22) PCT Filed: Jun. 26, 2019
(86) PCT No.: PCT/GB2019/051797

§ 371 Date: Dec. 9, 2020

(30) Foreign Application Priority Data
Jun. 26, 2018 (GB) 1810449.7

(51) Int. Cl. C07D 241/04; C07D 213/20

(52) U.S. Cl. CPC C07D 241/04; C07D 213/20

(57) ABSTRACT

A process for the production of heterocyclic quaternary ammonium salts or hydroxides is disclosed. The process comprises a continuous hydrogenation step, in which an unsaturated heterocyclic amine is reacted with hydrogen to form a saturated heterocyclic amine; a first continuous N-alkylation step, in which the saturated heterocyclic amine is alkylated to produce an intermediate saturated heterocyclic amine having an increased degree of substitution compared to the saturated heterocyclic amine; and one or more further N-alkylation steps in which the intermediate saturated heterocyclic amine is N-alkylated to the heterocyclic quaternary ammonium salt or hydroxide. A process of producing a saturated heterocyclic amine is also disclosed. The process comprises reacting an unsaturated heterocyclic amine with hydrogen in a vapour phase reaction at a pressure of not more than 70 bar and a temperature in the range of from 150° C. to 350° C. A process of N-alkylating a saturated heterocyclic amine is also disclosed. The process comprises N-alkylating the saturated heterocyclic amine in a vapour phase reaction at a temperature of at least 2° C.

### FIELD OF THE INVENTION

The present invention relates to the hydrogenation of unsaturated heterocyclic amines to produce saturated heterocyclic amines. In particular, but not exclusively, the present invention relates to the hydrogenation of 3,5 lutidine to produce 3,5 dimethylpiperidine. The present invention further relates to the N-alkylation of saturated heterocyclic amines. In particular, but not exclusively, the present invention relates to the N-alkylation of 3,5 dimethylpiperidine to produce 1,3,5 trimethylpiperidine. The present invention further relates to production of heterocyclic quaternary ammonium salts or hydroxides. In particular, the present invention further relates to the production of 1,1,3,5 tetramethylpiperidinium salts or hydroxides

### BACKGROUND

The hydrogenation of unsaturated heterocyclic amines to produce saturated heterocyclic amines is an important chemical process, as is the subsequent N-alkylation of those saturated heterocyclic amines. For example, the hydrogenation and N-alkylation may be steps in the production of compounds such as heterocyclic quaternary ammonium salts or hydroxides, which are used templating agents in the production of zeolites. Clearly the production of zeolites is an important commercial process, and lowering the cost of the production of the templating agents can lower the overall cost of producing the zeolites. Saturated heterocyclic amines, such as piperidine or pyrrolidine and their derivatives, may also have utility in other areas, such as pharmaceuticals, agrochemicals and industrial and personal/consumer care products. Processes for producing such compounds cost effectively would therefore be advantageous.

An example is the production of AEI zeolite, which uses 1,1,3,5 tetramethylpiperidinium hydroxide as a templating agent. One route for the production of 1,1,3,5 tetramethylpiperidinium hydroxide involves the double-alkylation of 3,5 dimethylpiperidine, which may be produced by hydrogenation of 3,5 lutidine.

The hydrogenation is typically carried out in the liquid phase, and can suffer from problems with deactivation of the catalyst as explained for example in Catalysis: Science and Technology by John R. Anderson and Michel Boudart p 112-3. Managing that deactivation increases the cost of the hydrogenation and hence the overall cost of the production of the 1,1,3,5 tetramethylpiperidinium hydroxide. Hydrogenation of 3,5 lutidine to 3,5 dimethylpiperidine is described in CN106916097A, CN100424075C, CN101104146B, CN102091638B and WO2016149234A1.

The N-alkylation proceeds in two steps, with the first N-alkylation converting 3,5 dimethylpiperidine to 1,3,5 trimethylpiperidine and the second converting the 1,3,5 trimethylpiperidine to a 1,1,3,5 tetramethylpiperidinium salt. Both N-alkylation steps are typically carried out in the liquid phase, typically as a batch process. The first N-alkylation may, for example, be carried out as an in-situ liquid phase reaction at the same time as a liquid phase hydrogenation of 3,5 lutidine to 3,5 dimethylpiperidine. While combining these steps in such a way may appear attractive, in practice the liquid phase hydrogenation can suffer from problems with deactivation of the catalyst as explained above. The N-alkylation can also be carried out in a separate liquid phase batch reaction, for example using alkylating reagents such as dimethyl sulphate or dimethyl carbonate. Methyl iodide and methanol with a promoter, typically a halogen such as iodine can also be used. However, the reaction times for such systems are typically hours and the reagents may suffer from significant disadvantages. For example, dimethyl sulphate is toxic and carcinogenic and dimethyl carbonate, while more benign, can generate significant by-products. N-alkylation of 3,5 dimethylpiperidine is described in CN106883165A.

Handling the problems with the prior art hydrogenation and N-alkylation processes increases the overall cost of the production of 1,1,3,5 tetramethylpiperidinium hydroxide, which in turn pushes up the cost of the AEI zeolite. Similar cost considerations apply to the manufacture of other similar templating agents. There therefore remains a need for cost-effective processes of hydrogenating unsaturated heterocyclic amines such as 3,5 lutidine to saturated heterocyclic amines such as 3,5 dimethylpiperidine and of alkylating saturated heterocyclic amines such as alkylating 3,5 dimethylpiperidine to 1,3,5 trimethylpiperidine and 1,3,5 trimethylpiperidine to a 1,1,3,5 tetramethylpiperidinium salt.

U.S. Pat. Nos. 6,086,848A, 5,965,104A, 6,444,191B1, 5,968,474A, 9,206,052B2, WO2016166245A1 and WO2015028971A1 describe making heterocyclic quaternary ammonium salts or hydroxides for use as templating agents in the manufacture of zeolites.

Preferred embodiments of the present invention seek to overcome one or more of the above disadvantages of the prior art. In particular, preferred embodiments of the present invention seek to provide an improved process of hydrogenating unsaturated heterocyclic amines to produce saturated heterocyclic amines, an improved process for N-alkylating saturated heterocyclic amines and an improved process for producing heterocyclic quaternary ammonium salts or hydroxides.

### SUMMARY OF INVENTION

According to a first aspect of the invention, there is provided a process of producing a saturated heterocyclic amine, the process comprising reacting an unsaturated heterocyclic amine with hydrogen in a vapour phase reaction at a pressure of not more than 70 bar and a temperature in the range of from 150° C. to 350° C.

By carrying out the hydrogenation in the vapour phase, the applicant has found that the catalyst deactivation that troubles prior art liquid phase hydrogenations can be avoided. Without wishing to be bound by theory, the applicant believes that the mechanism of catalyst deactivation is that the saturated heterocyclic amine produced in the hydrogenation poisons the catalyst. This is thought to be due to the highly basic nitrogen atom forming a strong sigma bond with the associated catalyst metal, as opposed to the weaker pi bonding that occurs during catalysis of the hydrogenation step. The nitrogen to metal bond thus formed is so strong that the saturated heterocyclic amine product stays absorbed on the catalyst, chemically blocking the active site. In prior art liquid phase hydrogenations, the catalyst typically needs maintaining by using an acid medium for the reaction, or by regenerating the catalyst with an acid wash, both of which add to the cost and complexity of the process. The vapour phase process of the present invention, by contrast, solves the deactivation issue. This is thought to be because the vapour phase reaction of the present invention reduces the residence time of the reactants and products on the catalyst, reduces mass transfer limitations of the hydrogen migrating to the catalyst, thus improving the equilibrium surface concentration of hydrogen, and/or reduces the propensity of the saturated heterocyclic amine to act as a Bronsted base. Moreover, the applicant has found that the vapour phase reaction can be carried out at pressures and temperatures that can readily be achieved in a cost-effective manner and without need for highly specialised, and therefore expensive, equipment. As a result, the invention allows a significant reduction in the cost of hydrogenating unsaturated heterocyclic amines to saturated heterocyclic amines.

Preferably the pressure is not more than 80 bar, more preferably not more than 70 bar. Lower pressures may be less costly to maintain and may reduce the cost of associated equipment. The pressure is preferably at least 1 bar, more preferably at least 10 bar and even more preferably at least 20 bar. Such pressures may improve the reaction rate and reduce the volume of gases passing through the reaction, thus also saving costs.

Preferably the temperature is not more than 300° C., more preferably not more than 270° C., even more preferably not more than 250° C. and yet more preferably not more than 220° C. Lower temperatures may reduce costs both in heating gases and in equipment costs. Preferably the temperature is at least 170° C. and more preferably at least 190° C. Such temperatures may ensure adequate activity and conversion.

Reduced residence times may be particularly advantageous for helping the vapour phase hydrogenation avoid the catalyst deactivation problems that affect liquid phase hydrogenations. The residence time may be described using the Gas Hourly Space Velocity (GHSV), which will be familiar to the skilled person and is defined as the volumetric flow rate of gaseous unsaturated heterocyclic amine into the reactor divided by the volume of catalyst in the reactor. Preferably the GHSV is at least 50 h⁻¹, more preferably at least 80 h⁻¹, and yet more preferably at least 90 h⁻¹ and even more preferably at least 95 h⁻¹. Preferably the GHSV is not more than 350 h⁻¹, more preferably not more than 320 h⁻¹ and yet more preferably not more than 300 h⁻¹. Where the unsaturated heterocyclic amine is fed as a liquid to a vaporiser, it may also be convenient to describe the throughput of unsaturated heterocyclic amine in terms of a Liquid Hourly Space Velocity (LHSV) defined as the volumetric flowrate of liquid unsaturated heterocyclic amine to the vaporiser divided by the volume of catalyst in the reactor. Preferably the LHSV is at least 0.3 h⁻¹, and more preferably at least 0.5 h⁻¹. Preferably the LHSV is not more than 3 h⁻¹, more preferably not more than 2 h⁻¹, even more preferably not more than 1.5 h⁻¹.

The presence of large excesses of hydrogen in a vapour phase reaction may also be advantageous in helping the vapour phase hydrogenation avoid the catalyst deactivation problems that affect liquid phase hydrogenations. Thus, the unsaturated heterocyclic amine and the hydrogen are preferably reacted at a molar ratio of at least 200 moles of hydrogen per mole of unsaturated heterocyclic amine and preferably at least 400 moles of hydrogen per mole of unsaturated heterocyclic amine. The unsaturated heterocyclic amine and the hydrogen are preferably reacted at a molar ratio of not more than 1000 moles of hydrogen per mole of unsaturated heterocyclic amine, preferably not more than 800 moles of hydrogen per mole of unsaturated heterocyclic amine.

Preferably the unsaturated heterocyclic amine is an unsaturated heterocyclic amine in which the nitrogen atom of the amine group is part of the heterocyclic ring. The unsaturated heterocyclic amine may be aromatic or aliphatic. For example, the unsaturated heterocyclic amine preferably comprises a pyridine ring or a pyrrole ring. Preferably the unsaturated heterocyclic amine comprises a single pyridine ring or a single pyrrole ring. For example, the unsaturated heterocyclic amine is preferably selected from the group consisting of: pyridine, 2-methyl pyridine, 3-methyl pyridine, 4-methyl pyridine, 2,6 lutidine, 3,5 lutidine, 2,4 lutidine, pyrrole, 2-methyl pyrrole, 3-methyl pyrrole, 2,4-dimethylpyrrole, and 2,5-dimethylpyrrole.

The skilled person will understand that the saturated heterocyclic amine is the compound resulting from saturation of the unsaturated heterocyclic amine. Thus, for example, the saturated heterocyclic amine preferably comprises a piperidine ring or a pyrrolidine ring. Preferably the saturated heterocyclic amine comprises a single piperidine ring or a single pyrrolidine ring. For example, the saturated heterocyclic amine is preferably selected from the group consisting of: piperidine, 2-methyl piperidine, 3-methyl piperidine, 4-methyl piperidine, 2,6 dimethylpiperidine, 3,5 dimethylpiperidine, pyrrolidine, 2-methyl pyrrolidine, 3-methyl pyrrolidine, 2,4-dimethyl pyrrolidine, and 2,5-dimethyl pyrrolidine. Preferably the saturated heterocyclic amine is a fully saturated heterocyclic amine.

Preferably the unsaturated heterocyclic amine is 3,5 lutidine and the saturated heterocyclic amine is 3,5 dimethylpiperidine. The hydrogenation of 3,5 lutidine to 3,5 dimethylpiperidine may be an important step in the production of 1,1,3,5 tetramethylpiperidinium hydroxide, which can be used as a templating agent in the production of AEI zeolite. By reducing the cost of the hydrogenation, the cost of the templating agent production can be reduced and ultimately therefore the cost of producing the zeolite is reduced.

The reaction will be carried out in a reactor. Preferably the reactor is a continuous reactor, in that the hydrogen and the unsaturated heterocyclic amine are continuously fed to the reactor and product, including the saturated heterocyclic amine, is continuously withdrawn from the reactor. Depending on the type of the reactor, there may be temperature variation across the reactor. In such embodiments, the temperature at the inlet of the reactor is defined as the reaction temperature. The temperature rise across the reactor is preferably not more than 200° C., preferably not more than 150° C. and more preferably not more than 100° C. The process may thus comprise feeding gaseous unsaturated heterocyclic amine and hydrogen gas to a reactor, reacting the unsaturated heterocyclic amine with the hydrogen in a vapour phase reaction in the reactor, wherein the reactor pressure is not more than 70 bar and the reactor inlet temperature is in the range of from 150° C. to 350° C. and withdrawing a product stream comprising saturated heterocyclic amine from the reactor. Preferably the feeding and withdrawing are continuous, by which the skilled person will understand that the process is a continuous rather than batch process. The gaseous unsaturated heterocyclic amine and hydrogen gas may be fed together or separately to the reactor. In a particularly preferred embodiment, liquid unsaturated heterocyclic amine is fed to a vaporiser where it is vaporised into a stream of hydrogen gas to create a mixed stream of hydrogen gas and gaseous unsaturated heterocyclic amine that is then fed to the reactor.

The catalyst may, for example, comprise one or more of nickel, ruthenium, rhodium or copper, for example on a support comprising one or more of alumina or silica. Preferably the catalyst comprises nickel as nickel catalysts have been found by the applicant to be effective catalysts and reasonably priced for the process of the invention. For example, the catalyst may be HTC500RP available from Johnson Matthey Plc. Such a catalyst may produce high levels of activity and conversion over a long lifetime. In some embodiments, it may be preferred to use a shape-selective catalyst to favour one product isomer over another. For example, where the unsaturated heterocyclic amine is 3,5 lutidine, a shape-selective catalyst may be used to favour the 3,5-cis dimethylpiperidine isomer.

According to a second aspect of the invention, there is provided a process of N-alkylating a saturated heterocyclic amine, the process comprising N-alkylating the saturated heterocyclic amine in a vapour phase reaction at a temperature of at least 220° C.

By carrying out the N-alkylation in the vapour phase, the applicant has found that the reaction proceeds in a matter of seconds, instead of hours, and the cost of the N-alkylation can be significantly reduced. This makes it possible, for example, to reduce the overall cost of producing templating agents for zeolite production. The applicant has also found that such a vapour phase N-alkylation proceeds with good conversion and selectivity and with low catalyst deactivation. Fewer unwanted by-products are formed compared to liquid-phase batch reactions. Such results contribute to the reduced cost of the process of the invention when compared with the prior art.

Preferably the process comprises reacting the saturated heterocyclic amine with dialkyl ether, preferably diethyl ether or dimethyl ether and most preferably dimethyl ether. The dialkyl ether may be fed directly to the reactor or may be generated in situ by the etherification of alkanol. For example, diethyl ether may be fed directly to the reactor or may be generated in situ by the etherification of ethanol. In a most preferable example, dimethyl ether may be fed directly to the reactor or may be generated in situ by the etherification of methanol. Dimethyl ether may advantageously be safer than alkylating agents such as dimethyl sulphate and may result in fewer by products than alkylating agents such as dimethyl carbonate.

Preferably the pressure is not more than 100 bar, preferably not more than 50 bar, more preferably not more than 35 bar and most preferably not more than 20 bar. Lower pressures may be less costly to maintain and may reduce the cost of associated equipment. The pressure is preferably at least 1 bar, more preferably at least 2 bar and even more preferably at least 5 bar. Such pressures may improve the reaction rate and volume of gases passing through the reaction, thus also saving costs.

Preferably the temperature is at least 250° C. and more preferably at least 280° C. Such temperatures may ensure adequate activity and conversion. Preferably the temperature is not more than 500° C., more preferably not more than 400° C., even more preferably not more than 350° C. and yet more preferably not more than 320° C. Lower temperatures may reduce costs both in heating gases and in equipment costs.

A particular advantage of the present invention may be that the residence time is reduced. The residence time may be described using the Gas Hourly Space Velocity (GHSV), which will be familiar to the skilled person and is defined as the volumetric flow rate of gaseous saturated heterocyclic amine into the reactor divided by the volume of catalyst in the reactor. Preferably the GHSV is at least 20 h⁻¹, more preferably at least 30 h⁻¹. Preferably the GHSV is not more than 300 h⁻¹, more preferably not more than 250 h⁻¹, even more preferably not more than 200 h⁻¹. Where the saturated heterocyclic amine is fed as a liquid to a vaporiser, it may also be convenient to describe the throughput of saturated heterocyclic amine in terms of a Liquid Hourly Space Velocity (LHSV) defined as the volumetric flowrate of liquid saturated heterocyclic amine to the vaporiser divided by the volume of catalyst in the reactor. Preferably the LHSV is not more than 2.0 h⁻¹, preferably not more than 1.5 h⁻¹, more preferably the LHSV is not more than 1.2 h⁻¹, most preferably the LHSV is not more than 1.1 h⁻¹. Preferably the LHSV is at least 0.1 h⁻¹, and more preferably at least 0.2 h⁻¹.

Where the dialkyl ether, such as diethyl ether or, most preferably, dimethyl ether, is generated in situ by the etherification of alkanol, such as the etherification of ethanol to produce diethyl ether or, most preferably, the etherification of methanol to produce dimethyl ether, the saturated heterocyclic amine and the alkanol are preferably fed to the process at a molar ratio of at least 1 mole of alkanol per mole of saturated heterocyclic amine, more preferably at least 1.2 moles of alkanol per mole of saturated heterocyclic amine, even more preferably at least 1.4 moles of alkanol per mole of saturated heterocyclic amine. The saturated heterocyclic amine and the alkanol are preferably fed to the process at a molar ratio of not more than 20 moles of alkanol per mole of saturated heterocyclic amine. The saturated heterocyclic amine and the alkanol are more preferably fed to the process at a molar ratio of not more than 10 moles of alkanol per mole of saturated heterocyclic amine. Such ratios may be particularly preferable where the alkanol is methanol and the dialkyl ether is dimethyl ether and may provide sufficient excess of dialkyl ether to promote a rapid reaction, while not unnecessarily increasing the volume of gases flowing through the reactor and thus the size and cost of the reactor.

Preferably the saturated heterocyclic amine is a saturated heterocyclic amine in which the nitrogen atom of the amine group is part of the heterocyclic ring. The saturated heterocyclic amine may be aromatic or aliphatic. Thus, for example, the saturated heterocyclic amine preferably comprises a piperidine ring or a pyrrolidine ring. Preferably the saturated heterocyclic amine comprises a single piperidine ring or a single pyrrolidine ring. For example, the saturated heterocyclic amine is preferably selected from the group consisting of: piperidine, 2-methyl piperidine, 3-methyl piperidine, 4-methyl piperidine, 2,6 dimethylpiperidine, 3,5 dimethylpiperidine, pyrrolidine, 2-methyl pyrrolidine, 3-methyl pyrrolidine, 2,4-dimethyl pyrrolidine, and 2,5-dimethyl pyrrolidine. Preferably the saturated heterocyclic amine is a fully saturated heterocyclic amine. The skilled person will understand that the N-alkylation increases the degree of saturation of the saturated heterocyclic amine, for example converting a primary amine into a secondary amine, a secondary amine into a tertiary amine or a tertiary amine into a quaternary ammonium salt or hydroxide. Preferably the N-alkylation is a methylation, in that the degree of substitution is increased by the addition of a methyl group, or an ethylation, in that the degree of substitution is increased by the addition of an ethyl group. Most preferably the N-alkylation is a methylation. Preferably the saturated heterocyclic amine is 3,5 dimethylpiperidine and the N-alkylation product is 1,3,5 trimethylpiperidine. The N-alkylation of 3,5 dimethylpiperidine to 1,3,5 trimethylpiperidine may be an important step in the production of 1,1,3,5 tetramethylpiperidinium hydroxide, which can be used as a templating agent in the production of AEI zeolite. By reducing the cost of the N-alkylation, the cost of the templating agent production can be reduced and ultimately therefore the cost of producing the zeolite is reduced.

The reaction will be carried out in a reactor. Preferably the reactor is a continuous reactor, in that the saturated heterocyclic amine is continuously fed to the reactor and product, including the N-alkylation product, is continuously withdrawn from the reactor. Depending on the type of the reactor, there may be temperature variation across the reactor. In such embodiments, the temperature at the inlet of the reactor is defined as the reaction temperature. The temperature rise across the reactor is preferably not more than 200° C., preferably not more than 150° C. and more preferably not more than 100° C. The process may thus comprise feeding gaseous saturated heterocyclic amine to a reactor, N-alkylating the saturated heterocyclic amine in a vapour phase reaction in the reactor to produce an N-alkylation product, wherein the reactor inlet temperature is at least 220° C., and withdrawing a product stream comprising the N-alkylation product from the reactor. The N-alkylation product has a greater degree of substitution than the saturated heterocyclic amine. Preferably the feeding and withdrawing are continuous, by which the skilled person will understand that the process is a continuous rather than batch process. In one embodiment, the gaseous saturated heterocyclic amine and alkylating reagent, for example methanol, ethanol, diethyl ether or dimethyl ether, preferably methanol or ethanol, more preferably methanol or dimethyl ether, and yet more preferably methanol, may be fed together to the reactor. In a particularly preferred embodiment, liquid saturated heterocyclic amine is fed to a vaporiser where it is vaporised along with alkylating reagent, for example methanol, ethanol, diethyl ether or dimethyl ether, preferably methanol or ethanol, more preferably methanol or dimethyl ether, and yet more preferably methanol, to create a mixed gaseous stream of alkylating reagent, for example methanol, ethanol, diethyl ether or dimethyl ether, preferably methanol or ethanol, more preferably methanol or dimethyl ether, and yet more preferably methanol, and saturated heterocyclic amine that is then fed to the reactor.

Preferably the process is carried out downstream of a hydrogenation of unsaturated heterocyclic amine to produce the saturated heterocyclic amine. Thus, a process may be provided for hydrogenating an unsaturated heterocyclic amine to a saturated heterocyclic amine and N-alkylating the saturated heterocyclic amine. Preferably the hydrogenation is a vapour phase hydrogenation. Preferably the hydrogenation is a hydrogenation according to the first aspect of the invention. The saturated heterocyclic amine may be collected from the hydrogenation, for example by cooling and collecting as a condensed liquid, optionally with excess hydrogen being removed and, for example, recycled. The collected saturated heterocyclic amine may then be fed to the N-alkylation as described above. Such a process may be advantageous in that the hydrogenation can run with a high level of excess hydrogen, which is then removed before the N-alkylation so that the N-alkylation reactor does not need to be sized for handling the excess hydrogen. However, in some embodiments the gaseous products from the hydrogenation, comprising the saturated heterocyclic amine and hydrogen, are kept in the gas phase and fed to the N-alkylation reactor. Such a process may efficiently transfer the saturated heterocyclic amine, without the need for cooling and separating. Preferably the gaseous products are heated before being fed to the N-alkylation reactor. Preferably alkylating reagent, for example methanol, ethanol, diethyl ether or dimethyl ether, preferably methanol or ethanol, more preferably methanol or dimethyl ether, and yet more preferably methanol, is also added to the N-alkylation reactor, for example by being added to the gaseous products before they are fed to the N-alkylation reactor and preferably before they are heated. Preferably the hydrogenation and the N-alkylation processes are continuous and are combined to create a continuous process for hydrogenation and N-alkylation.

The catalyst may for example comprise one or more of nickel, ruthenium, rhodium or copper, for example on a support comprising one or more of alumina or silica. In some embodiments, the catalyst comprises nickel. For example, the catalyst may be an HTC Nickel series catalyst available from Johnson Matthey. However, most preferably the catalyst comprises an alumina, most preferably a γ-alumina. Optionally the alumina catalyst also comprises silica, for example at least 1 wt % silica, preferably at least 2 wt % silica and more preferably at least 2.5 wt % silica. Preferably the alumina catalyst comprises not more than 5 wt % silica, more preferably not more than 4 wt % silica and more preferably not more than 3.5 wt % silica. Example catalysts include Extral 12, Extral 25 or HTC AQ available from Johnson Matthey. Such a catalyst may produce high levels of activity and conversion over a long lifetime.

According to a third aspect of the invention, there is provided a process for the production of heterocyclic quaternary ammonium salts or hydroxides, the process comprising a continuous hydrogenation step, preferably in the vapour phase, in which an unsaturated heterocyclic amine is reacted with hydrogen to form a saturated heterocyclic amine; a first continuous N-alkylation step, preferably in the vapour phase, in which the saturated heterocyclic amine is N-alkylated to produce an intermediate saturated heterocyclic amine having an increased degree of substitution compared to the saturated heterocyclic amine; and one or more further N-alkylation steps, preferably continuous, in which the intermediate saturated heterocyclic amine is N-alkylated to the heterocyclic quaternary ammonium salt or hydroxide. Optionally the process further comprises a counter ion swap step, preferably continuous, in which a first counter ion on the heterocyclic quaternary ammonium salt or hydroxide is exchanged for a second counter ion.

Prior art processes have typically carried out hydrogenation and N-alkylation steps as batch processes in the liquid phase. By carrying out the hydrogenation and first N-alkylation as continuous processes, preferably in the vapour phase, the overall cost of the process may advantageously be decreased. The cost decrease may come from a combination of factors, for example including improved catalyst lifetime, improved reaction rates, less manual intervention and more efficient transfer of materials between stages of the process.

The hydrogenation step may be according to the first aspect of the invention. The first N-alkylation step may be according to the second aspect of the invention. It will be appreciated that features described in relation to those aspects of the invention may be equally advantageous and preferable in the present aspect of the invention.

Preferably the unsaturated heterocyclic amine is an unsaturated heterocyclic amine in which the nitrogen atom of the amine group is part of the heterocyclic ring. The unsaturated heterocyclic amine may be aromatic or aliphatic. For example, the unsaturated heterocyclic amine preferably comprises a pyridine ring or a pyrrole ring. Preferably the unsaturated heterocyclic amine comprises a single pyridine ring or a single pyrrole ring. For example, the unsaturated heterocyclic amine is preferably selected from the group consisting of: pyridine, 2-methyl pyridine, 3-methyl pyridine, 4-methyl pyridine, 2,6 lutidine, 3,5 lutidine, pyrrole, 2-methyl pyrrole, 3-methyl pyrrole, 2,4-dimethylpyrrole, and 2,5-dimethylpyrrole.

The skilled person will understand that the saturated heterocyclic amine is the compound resulting from saturation of the unsaturated heterocyclic amine. Thus, for example, the saturated heterocyclic amine preferably comprises a piperidine ring or a pyrrolidine ring. Preferably the saturated heterocyclic amine comprises a single piperidine ring or a single pyrrolidine ring. For example, the saturated heterocyclic amine is preferably selected from the group consisting of: piperidine, 2-methyl piperidine, 3-methyl piperidine, 4-methyl piperidine, 2,6 dimethylpiperidine, 3,5 dimethylpiperidine, pyrrolidine, 2-methyl pyrrolidine, 3-methyl pyrrolidine, 2,4-dimethyl pyrrolidine, and 2,5-dimethyl pyrrolidine. Preferably the saturated heterocyclic amine is a fully saturated heterocyclic amine.

The skilled person will understand that the intermediate saturated heterocyclic amine and the heterocyclic quaternary ammonium salt or hydroxide will correspond to the saturated heterocyclic amine substituted with alkyl groups corresponding to the N-alkylation agent used. For example, the alkyl groups may be methyl groups or ethyl groups. The alkyl groups will be substituted on the nitrogen atom of the amine.

Thus, for example, the intermediate saturated heterocyclic amine is preferably a saturated heterocyclic alkylamine. It will be understood that the intermediate saturated heterocyclic amine is an alkylamine in that the nitrogen atom of the amine has an alkyl group attached, but that further alkyl groups can be attached elsewhere, for example at other locations on the ring. Thus, the intermediate saturated heterocyclic amine is optionally an alkylated saturated heterocyclic alkylamine. More preferably the intermediate saturated heterocyclic amine is an, optionally alkylated, saturated heterocyclic methylamine or ethylamine. More preferably the intermediate saturated heterocyclic amine is an, optionally methylated or ethylated, saturated heterocyclic methylamine or ethylamine. Most preferably the intermediate saturated heterocyclic amine is an, optionally methylated, saturated heterocyclic methylamine. The intermediate saturated heterocyclic amine preferably comprises a piperidine ring or a pyrrolidine ring. Preferably the intermediate saturated heterocyclic amine comprises a single piperidine ring or a single pyrrolidine ring. For example, the intermediate saturated heterocyclic amine is preferably selected from the group consisting of: 1-methylpiperidine, 1,2-dimethyl piperidine, 1,3-dimethyl piperidine, 1,4-dimethyl piperidine, 1,2,6 trimethylpiperidine, 1,3,5 trimethylpiperidine, 1-methylpyrrolidine, 1,2-dimethyl pyrrolidine, 1,3-dimethyl pyrrolidine, 1,2,4-trimethyl pyrrolidine, and 1,2,5-trimethyl pyrrolidine.

The heterocyclic quaternary ammonium salt or hydroxide is preferably a heterocyclic quaternary dialkylammonium salt or hydroxide. It will be understood that the heterocyclic quaternary dialkylammonium salt or hydroxide is a dialkylammonium salt or hydroxide in that the nitrogen atom of the ammonium has two alkyl groups attached, but that further alkyl groups can be attached elsewhere, for example at other locations on the ring. Thus, the quaternary dialkylammonium salt or hydroxide is optionally an alkylated quaternary dialkylammonium salt or hydroxide. More preferably the quaternary dialkylammonium salt or hydroxide is an, optionally alkylated, heterocyclic quaternary dimethylammonium, methylethylammonium or diethylammonium salt or hydroxide. More preferably the quaternary dialkylammonium salt or hydroxide is an, optionally methylated or ethylated, heterocyclic quaternary dimethylammonium, methylethylammonium or diethylammonium salt or hydroxide. More preferably the quaternary dialkylammonium salt or hydroxide is an, optionally methylated, heterocyclic quaternary dimethylammonium salt or hydroxide. The heterocyclic quaternary ammonium salt or hydroxide preferably comprises a piperidinium ring or a pyrrolidinium ring. Preferably the heterocyclic quaternary ammonium salt or hydroxide comprises a single piperidinium ring or a single pyrrolidinium ring. For example, the heterocyclic quaternary ammonium salt or hydroxide is preferably selected from the group consisting of: 1,1-dimethylpiperidinium salt or hydroxide, 1,1,2-trimethylpiperidinium salt or hydroxide, 1,1,3-trimethylpiperidinium salt or hydroxide, 1,1,4-trimethylpiperidinium salt or hydroxide, 1,1,2,6 tetramethylpiperidinium salt or hydroxide, 1,1,3,5 tetramethylpiperidinium salt or hydroxide, 1,1-dimethylpyrrolidinium salt or hydroxide, 1,1,2-trimethylpyrrolidinium salt or hydroxide, 1,1,3-trimethylpyrrolidinium salt or hydroxide, 1,1,2,4-tetramethyl pyrrolidine salt or hydroxide, and 1,1,2,5-tetramethyl pyrrolidine salt or hydroxide.

Preferably the unsaturated heterocyclic amine is 3,5 lutidine, the saturated heterocyclic amine is 3,5 dimethylpiperidine, the intermediate saturated heterocyclic amine is 1,3,5 trimethylpiperidine and the heterocyclic quaternary ammonium salt or hydroxide is 1,1,3,5 tetramethylpiperidinium hydroxide. 1,1,3,5 tetramethylpiperidinium hydroxide can be used as a templating agent in the production of AEI zeolite. By reducing the cost of producing the 1,1,3,5 tetramethylpiperidinium hydroxide, the cost of producing the zeolite is reduced.

The hydrogenation step may comprise continuously feeding the unsaturated heterocyclic amine and hydrogen, preferably in the gas phase to a reactor. The reactor is a continuous reactor, in that the hydrogen and the unsaturated heterocyclic amine are continuously fed to the reactor and product, including the saturated heterocyclic amine, is continuously withdrawn from the reactor. Depending on the type of the reactor, there may be temperature variation across the reactor. In such embodiments, the temperature at the inlet of the reactor is defined as the reaction temperature. The temperature rise across the reactor is preferably not more than 200° C., preferably not more than 150° C. and more preferably not more than 100° C. The feeding and withdrawing are continuous, by which the skilled person will understand that the process is a continuous rather than batch process. The gaseous unsaturated heterocyclic amine and hydrogen gas may be fed together or separately to the reactor. In a particularly preferred embodiment, liquid unsaturated heterocyclic amine is fed to a vaporiser where it is vaporised into a stream of hydrogen gas to create a mixed stream of hydrogen gas and gaseous unsaturated heterocyclic amine that is then fed to the reactor.

Preferably the pressure of the hydrogenation step is not more than 70 bar, preferably not more than 60 bar, more preferably not more than 50 bar. Lower pressures may be less costly to maintain and may reduce the cost of associated equipment. The pressure is preferably at least 1 bar, more preferably at least 10 bar and even more preferably at least 20 bar. Such pressures may improve the reaction rate and volume of gases passing through the reaction, thus also saving costs.

Preferably the temperature of the hydrogenation step is not more than 350° C., preferably not more than 300° C., more preferably not more than 270° C., even more preferably not more than 250° C. and yet more preferably not more than 220° C. Lower temperatures may reduce costs both in heating gases and in equipment costs. Preferably the temperature is at least 150° C., preferably at least 170° C. and more preferably at least 190° C. Such temperatures may ensure adequate activity and conversion.

Where the hydrogenation step is a vapour phase hydrogenation step, reduced residence times may be particularly advantageous for helping the vapour phase hydrogenation step avoid the catalyst deactivation problems that affect liquid phase hydrogenations. The residence time may be described using the Gas Hourly Space Velocity (GHSV), which will be familiar to the skilled person and is defined as the volumetric flow rate of gaseous unsaturated heterocyclic amine into the reactor divided by the volume of catalyst in the reactor. Preferably the GHSV is at least 50 h⁻¹, more preferably at least 80 h⁻¹, and yet more preferably at least 90 h⁻¹ and even more preferably at least 95 h⁻¹. Preferably the GHSV is not more than 350 h⁻¹, more preferably not more than 320 h⁻¹ and yet more preferably not more than 300 h⁻¹. Where the unsaturated heterocyclic amine is fed as a liquid to a vaporiser, it may also be convenient to describe the throughput of unsaturated heterocyclic amine in terms of a Liquid Hourly Space Velocity (LHSV) defined as the volumetric flowrate of liquid unsaturated heterocyclic amine to the vaporiser divided by the volume of catalyst in the reactor. Preferably the LHSV is at least 0.3 h⁻¹, and more preferably at least 0.5 h⁻¹. Preferably the LHSV is not more than 3 h⁻¹, more preferably not more than 2 h⁻¹, even more preferably not more than 1.5 h⁻¹ and yet more preferably not more than 1 h⁻¹.

Where the hydrogenation step is a vapour phase hydrogenation step, the presence of large excesses of hydrogen in a vapour phase reaction may also be advantageous in helping the vapour phase hydrogenation avoid the catalyst deactivation problems that affect liquid phase hydrogenations. Thus, the unsaturated heterocyclic amine and the hydrogen are preferably reacted at a molar ratio of at least 200 moles of hydrogen per mole of unsaturated heterocyclic amine and preferably at least 400 moles of hydrogen per mole of unsaturated heterocyclic amine. The unsaturated heterocyclic amine and the hydrogen are preferably reacted at a molar ratio of not more than 1000 moles of hydrogen per mole of unsaturated heterocyclic amine, preferably not more than 800 moles of hydrogen per mole of unsaturated heterocyclic amine.

The first N-alkylation step is carried out in a continuous reactor, in that the saturated heterocyclic amine is preferably continuously fed to the reactor and product, including the intermediate saturated heterocyclic amine, is continuously withdrawn from the reactor. Depending on the type of the reactor, there may be temperature variation across the reactor. In such embodiments, the temperature at the inlet of the reactor is defined as the reaction temperature. The temperature rise across the reactor is preferably not more than 200° C., preferably not more than 150° C. and more preferably not more than 100° C. The feeding and withdrawing are continuous, by which the skilled person will understand that the process is a continuous rather than batch process. In one embodiment, gaseous saturated heterocyclic amine and alkylating reagent, for example methanol, ethanol, diethyl ether or dimethyl ether, preferably methanol or ethanol, more preferably methanol or dimethyl ether, and yet more preferably methanol, may be fed together to the reactor. In a particularly preferred embodiment, liquid saturated heterocyclic amine is fed to a vaporiser where it is vaporised along with alkylating reagent, for example methanol, ethanol, diethyl ether or dimethyl ether, preferably methanol or ethanol, more preferably methanol or dimethyl ether, and yet more preferably methanol, to create a mixed gaseous stream of alkylating reagent, for example methanol, ethanol, diethyl ether or dimethyl ether, preferably methanol or ethanol, more preferably methanol or dimethyl ether, and yet more preferably methanol, and saturated heterocyclic amine that is then fed to the reactor.

Preferably the saturated heterocyclic amine is collected from the hydrogenation step, for example by cooling and collecting the saturated heterocyclic amine as a condensed liquid, optionally with excess hydrogen being removed and, for example, recycled. The collection is preferably carried out continuously, for example by passing a product stream from the hydrogenation step to a knock-out pot in which the saturated heterocyclic amine is condensed and separated from hydrogen in the product stream. The collected saturated heterocyclic amine may then be fed to the first N-alkylation step as described above. The hydrogen may be recycled to the hydrogenation step. Such a process may be advantageous in that the hydrogenation can run with a high level of excess hydrogen, which is then removed before the first N-alkylation step so that the reactor in the first N-alkylation step does not need to be sized for handling the excess hydrogen.

However, in some embodiments the products from the hydrogenation step, comprising the saturated heterocyclic amine and hydrogen, are fed directly to the first N-alkylation step, in that they are fed, optionally with intermediate heating or cooling, without any separation taking place. In some embodiments, where the hydrogenation and the first N-alkylation step are both in the vapour phase, the gaseous products from the hydrogenation step, comprising the saturated heterocyclic amine and hydrogen, are kept in the gas phase and fed to the N-alkylation step. Such a process may efficiently transfer the saturated heterocyclic amine, without the need for cooling and separating. Preferably the gaseous products are heated before being fed to the first N-alkylation step. Preferably alkylating reagent, for example methanol, ethanol, diethyl ether or dimethyl ether, preferably methanol or ethanol, more preferably methanol or dimethyl ether, and yet more preferably methanol, is also added to the first N-alkylation step, for example by being added to the gaseous products before they are fed to the first N-alkylation step and preferably before they are heated.

In some embodiments, the hydrogenation step and the first N-alkylation step are carried out in the same reactor. In such embodiments, the unsaturated heterocyclic amine reacts with hydrogen to form the saturated heterocyclic amine, which is then alkylated in the same reactor by reaction with an alkylating agent. Preferably the alkylating agent is dialkyl ether, such as diethyl ether or, most preferably, dimethyl ether, which may be generated in situ from alkanol, such as in situ generation of diethyl ether from ethanol or, most preferably, in situ generation of dimethyl ether from methanol. Thus, in some embodiments, gaseous unsaturated heterocyclic amine, hydrogen and alkylating reagent, for example methanol, ethanol, diethyl ether or dimethyl ether, preferably methanol or ethanol, more preferably methanol or dimethyl ether, and yet more preferably methanol, are fed to a reactor in which the hydrogenation step and the N-alkylation step take place. Preferably the unsaturated heterocyclic amine and the alkylating reagent, for example methanol, ethanol, diethyl ether or dimethyl ether, preferably methanol or ethanol, more preferably methanol or dimethyl ether, and yet more preferably methanol, are fed as a liquid to a vaporiser wherein they are vaporised into a stream comprising gaseous hydrogen. The resulting gaseous stream is then fed to the reactor.

Preferably the first N-alkylation step comprises reacting the saturated heterocyclic amine with dialkyl ether, preferably diethyl ether or dimethyl ether and most preferably dimethyl ether. The dialkyl ether may be fed directly to the reactor or may be generated in situ by the etherification of alkanol. For example, diethyl ether may be fed directly to the reactor or may be generated in situ by the etherification of ethanol. In a most preferable example, dimethyl ether may be fed directly to the reactor or may be generated in situ by the etherification of methanol. Dimethyl ether may advantageously be safer than alkylating agents such as dimethyl sulphate and may result in fewer by products that alkylating agents such as dimethyl carbonate.

Preferably the pressure is not more than 100 bar, preferably not more than 50 bar, more preferably not more than 35 bar and most preferably not more than 20 bar. Lower pressures may be less costly to maintain and may reduce the cost of associated equipment. The pressure is preferably at least 1 bar, more preferably at least 2 bar and even more preferably at least 5 bar. Such pressures may improve the reaction rate and volume of gases passing through the reaction, thus also saving costs.

Preferably the temperature is at least 220° C., preferably at least 250° C. and more preferably at least 280° C. Such temperatures may ensure adequate activity and conversion. Preferably the temperature is not more than 500° C., more preferably not more than 400° C., even more preferably not more than 350° C. and yet more preferably not more than 320° C. Lower temperatures may reduce costs both in heating gases and in equipment costs.

A particular advantage of the present invention may be that the residence time is reduced. The residence time may be described using the Gas Hourly Space Velocity (GHSV), which will be familiar to the skilled person and is defined as the volumetric flow rate of gaseous saturated heterocyclic amine into the reactor divided by the volume of catalyst in the reactor. Preferably the GHSV is at least 20 h⁻¹, more preferably at least 30 h⁻¹. Preferably the GHSV is not more than 200 h⁻¹, more preferably not more than 150 h⁻¹, even more preferably not more than 100 h⁻¹ and yet more preferably not more than 80 h⁻¹.

Where the saturated heterocyclic amine is fed as a liquid to a vaporiser, it may also be convenient to describe the throughput of saturated heterocyclic amine in terms of a Liquid Hourly Space Velocity (LHSV) defined as the volumetric flowrate of liquid saturated heterocyclic amine to the vaporiser divided by the volume of catalyst in the reactor. Preferably the LHSV is not more than 2.0 h⁻¹, preferably not more than 1.5 h⁻¹, more preferably the LHSV is not more than 1.2 h⁻¹, most preferably the LHSV is not more than 1.1 h⁻¹. Preferably the LHSV is at least 0.1 h⁻¹, and more preferably at least 0.2 h⁻¹.

Where the dialkyl ether, such as diethyl ether or, most preferably, dimethyl ether, is generated in situ by the etherification of alkanol, such as the etherification of ethanol to produce diethyl ether or, most preferably, the etherification of methanol to produce dimethyl ether, the saturated heterocyclic amine and the alkanol are preferably fed to the process at a molar ratio of at least 1 mole of alkanol per mole of saturated heterocyclic amine, more preferably at least 1.2 moles of alkanol per mole of saturated heterocyclic amine, even more preferably at least 1.5 moles of alkanol per mole of saturated heterocyclic amine, and yet more preferably at least 2 moles of alkanol per mole of saturated heterocyclic amine. The saturated heterocyclic amine and the alkanol are preferably fed to the process at a molar ratio of not more than 20 moles of alkanol per mole of saturated heterocyclic amine. The saturated heterocyclic amine and the alkanol are more preferably fed to the process at a molar ratio of not more than 10 moles of alkanol per mole of saturated heterocyclic amine. Such ratios may be particularly preferable where the alkanol is methanol and the dialkyl ether is dimethyl ether and may provide sufficient excess of dialkyl ether to promote a rapid reaction, while not unnecessarily increasing the volume of gases flowing through the reactor and thus the size and cost of the reactor.

The one or more further N-alkylation steps may be carried out in the liquid phase. That may be advantageous as the heterocyclic quaternary ammonium salt produced in the one or more further N-alkylation steps may have a low vapour pressure. The one or more further N-alkylation steps may be carried out as batch processes, but are preferably continuous. For example, the one or more further N-alkylation steps may be carried out continuously in one or more continuous stirred tank reactors.

The one or more further N-alkylation steps may be carried out by reacting the intermediate saturated heterocyclic amine with dimethyl carbonate or dimethyl sulphate. Reacting with dimethyl carbonate may be preferred, particularly where the first N-alkylation step is carried out by reacting the saturated heterocyclic amine with dimethyl ether. In the case where the dimethyl ether is produced in situ from methanol, water will be produced, which may react with the heterocyclic quaternary ammonium methyl carbonate produced by the N-alkylation with dimethyl carbonate to produce one or more of heterocyclic quaternary ammonium carbonate, heterocyclic quaternary ammonium hydrogen carbonate and heterocyclic quaternary ammonium hydroxide. The hydroxide may, for example, be the desired product and it is therefore advantageous to produce some hydroxide directly in the one or more further N-alkylation steps before any counter ion swap step is used.

It may be desirable to carry out a counter ion swap step, in which a first counter ion on the heterocyclic quaternary ammonium salt or hydroxide is exchanged for a second counter ion. For example, where the hydroxide is the desired product, the counter ion swap step may comprise reacting the heterocyclic quaternary ammonium salt with a suitable hydroxide species such as sodium or calcium hydroxide to form heterocyclic quaternary ammonium hydroxide and an organometallic salt, such as sodium or calcium methyl carbonate or methyl sulphate which can be removed to leave the final heterocyclic quaternary ammonium hydroxide product. The counter ion swap step is preferably carried out in the liquid phase. The counter ion swap step may be carried out as a batch process, but is preferably carried out as a continuous process, for greater efficiency and reduced cost, for example using a continuous stirred tank reactor or a suitable hydroxide ion exchange resin.

### DESCRIPTION OF THE DRAWINGS

Embodiments of the present invention will now be described, by way of example, and not in any limitative sense, with reference to the accompanying drawings, of which:

FIG. 1 is a flowsheet of a process according to the first aspect of the invention;

FIG. 2 is a graph of conversion and selectivity;

FIG. 3 is a graph of conversion and activity at a ratio of 800 moles hydrogen per mole of lutidine;

FIG. 4 is a graph of conversion and activity at a ratio of 600 moles hydrogen per mole of lutidine;

FIG. 5 is a graph of conversion and activity at a ratio of 400 moles hydrogen per mole of lutidine;

FIG. 6 is a graph of conversion and activity plotted against temperature;

FIG. 7 is a flowsheet of a process according to the second aspect of the invention;

FIG. 8 is a flowsheet of processes according to the first and second aspects of the invention;

FIG. 9 is a flowsheet of processes according to the first and second aspects of the invention;

FIG. 10 is a flowsheet of processes according to the third aspect of the invention;

FIG. 11 is a flowsheet of processes according to the first second and third aspects of the invention;

FIG. 12 is a graph of conversion against time;

FIG. 13 is a graph of conversion against pressure; and

FIG. 14 is a graph of conversion for 4 different catalysts.

### DETAILED DESCRIPTION

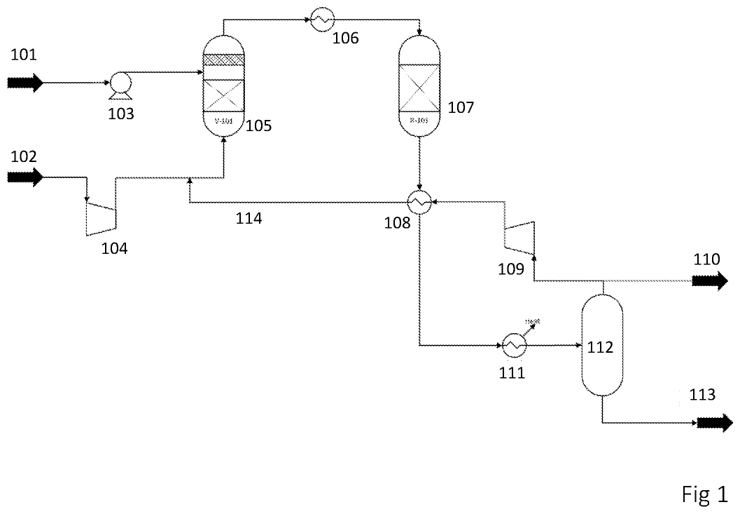

In FIG. 1, hydrogen gas 102 is compressed 104 and fed to a vaporiser 105. Unsaturated heterocyclic amine, in this embodiment 3,5 lutidine 101, is pumped 103 into the vaporiser 105, where it is vaporised into the hydrogen gas. The stream exiting the vaporiser 105 comprises gaseous 3,5 lutidine and hydrogen gas and is heated 106 and fed to a reactor 107. In the reactor 107 a vapour phase reaction between the 3,5 lutidine and the hydrogen takes place and 3,5 dimethylpiperidine is formed. Preferably the pressure in the reactor 107 is not more than 70 bar and the inlet temperature of the reactor 107 is preferably in the range of from 150° C. to 350° C. Product from the reactor 107, which includes the 3,5 dimethylpiperidine is cooled, first by heat exchange 108 with a hydrogen recycle stream 114 and then by cooling 111, before being fed to a hydrogen knock-out pot 112. In hydrogen knock out pot 112, the product 3,5 dimethylpiperidine condenses and is produced as liquid 3,5 dimethylpiperidine 113, while the hydrogen remains as a gas and is produced overhead. The hydrogen is either compressed 109 and recycled in hydrogen recycle stream 114, or it is purged 110.

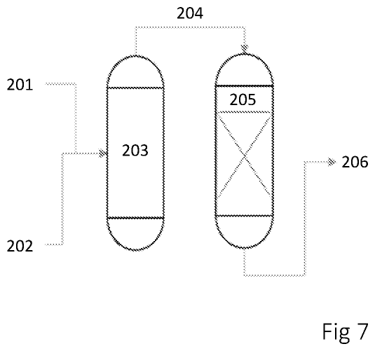

In FIG. 7, a feed comprising 3,5 dimethylpiperidine 201 and methanol 202 is fed to a vaporiser 203 in which the 3,5 dimethylpiperidine 201 and the methanol 202 are vaporised to form a mixed gas stream 204 comprising 3,5 dimethylpiperidine and methanol. The mixed gas stream 204 is fed to an N-alkylation reactor 205, where the methanol is converted to dimethyl ether and reacts with the 3,5 dimethylpiperidine, for example over an alumina catalyst, to produce 1,3,5 trimethylpiperidine, which is withdrawn in a product stream 206. The N-alkylation reactor 205 preferably has an inlet temperature of at least 220° C.

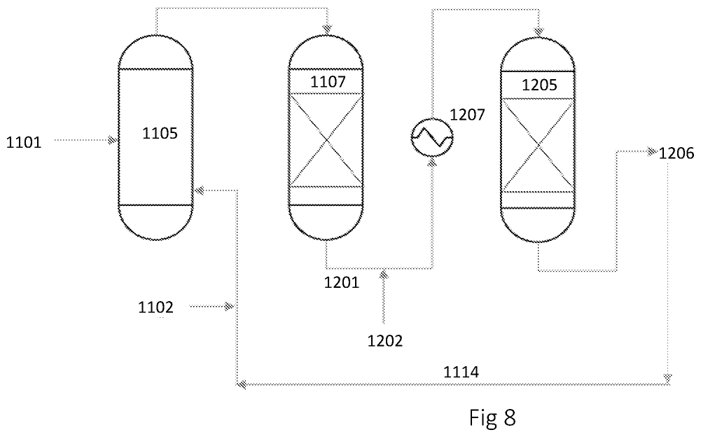

In FIG. 8, a stream comprising 3,5 lutidine 1101 is fed to a vaporiser 1105 where it is vaporised into a stream of hydrogen 1102. The resulting gas stream is fed to a hydrogenation reactor 1107, where the 3,5 lutidine is hydrogenated to 3,5 dimethylpiperidine, for example over a nickel catalyst. The pressure in the reactor 1107 is preferably not more than 70 bar and the inlet temperature of the reactor 1107 is preferably in the range of from 150° C. to 350° C. A product stream 1201 from the hydrogenation reactor 1107 is mixed with methanol 1202 and heated in heater 1207 before being passed to an N-alkylation reactor 1205. In the N-alkylation reactor 1205, the methanol forms dimethyl ether, which reacts with the 3,5 dimethylpiperidine, for example over an alumina catalyst, to produce 1,3,5 trimethylpiperidine, which is withdrawn in a product stream 1206. The N-alkylation reactor 1205 preferably has an inlet temperature of at least 220° C. Hydrogen is separated from the product stream 1206 and recycled via hydrogen recycle stream 1114.

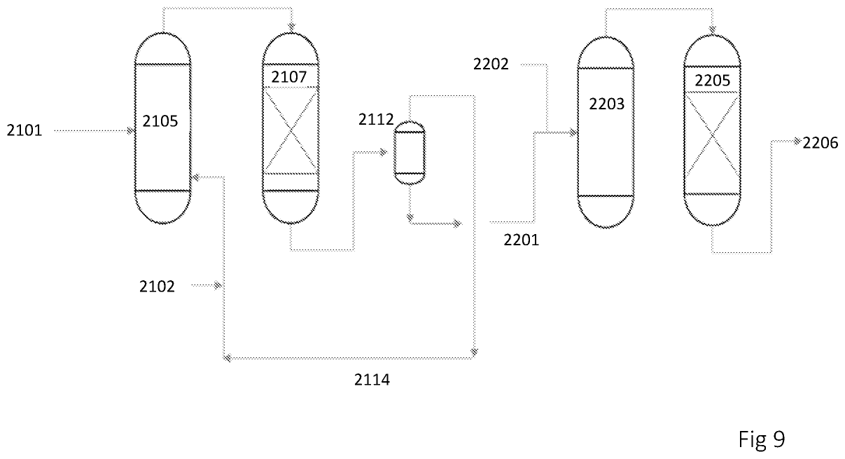

In FIG. 9, a stream of 3,5 lutidine 2101 is fed to a vaporiser 2105 where it is vaporised into a stream of hydrogen 2102. The resulting gas stream is fed to a hydrogenation reactor 2107, where the 3,5 lutidine is hydrogenated to 3,5 dimethylpiperidine, for example over a nickel catalyst. A product stream from the hydrogenation reactor 2107 is fed to a knock-out pot 2112, where hydrogen gas comes off overhead and is recycled via hydrogen recycle line 2114, and a 3,5 dimethylpiperidine stream 2201 comes off as a liquid. The 3,5 dimethylpiperidine stream 2201 is mixed with methanol 2202 and fed to a vaporiser 2203 before being passed to an N-alkylation reactor 2205. In the N-alkylation reactor 2205, the methanol forms dimethyl ether, which reacts with the 3,5 dimethylpiperidine, for example over an alumina catalyst, to produce 1,3,5 trimethylpiperidine, which is withdrawn in a product stream 2206. The N-alkylation reactor 2205 has an inlet temperature of at least 220° C.

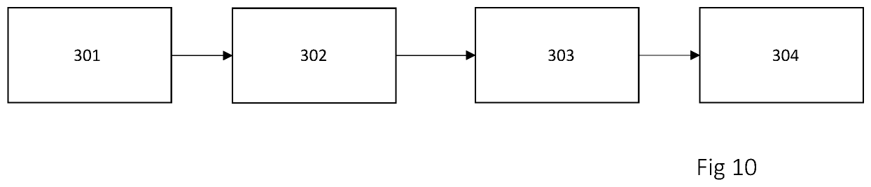

In FIG. 10 a process for producing 1,1,3,5 tetramethylpiperidinium hydroxide involves a hydrogenation step 301, in which 3,5 lutidine is hydrogenated to 3,5 dimethylpiperidine, a first N-alkylation step 302, in which the 3,5 dimethylpiperidine is alkylated to 1,3,5 trimethylpiperidine, a second N-alkylation step 303 in which the 1,3,5 trimethylpiperidine is alkylated to one or more of 1,1,3,5 tetramethylpiperidinium methyl carbonate, 1,1,3,5 tetramethylpiperidinium hydrogen carbonate and 1,1,3,5 tetramethylpiperidinium hydroxide, and a counter-ion swap step 304, in which 1,1,3,5 tetramethylpiperidinium methyl carbonate and 1,1,3,5 tetramethylpiperidinium hydrogen carbonate are converted to 1,1,3,5 tetramethylpiperidinium hydroxide. In a particular embodiment, the hydrogenation step 301 and the N-alkylation step 302 are continuous vapour phase reactions. The hydrogenation step 301 and the first N-alkylation step 302, may for example be as described in relation to any of FIGS. 1 to 9 or 11 to 14.

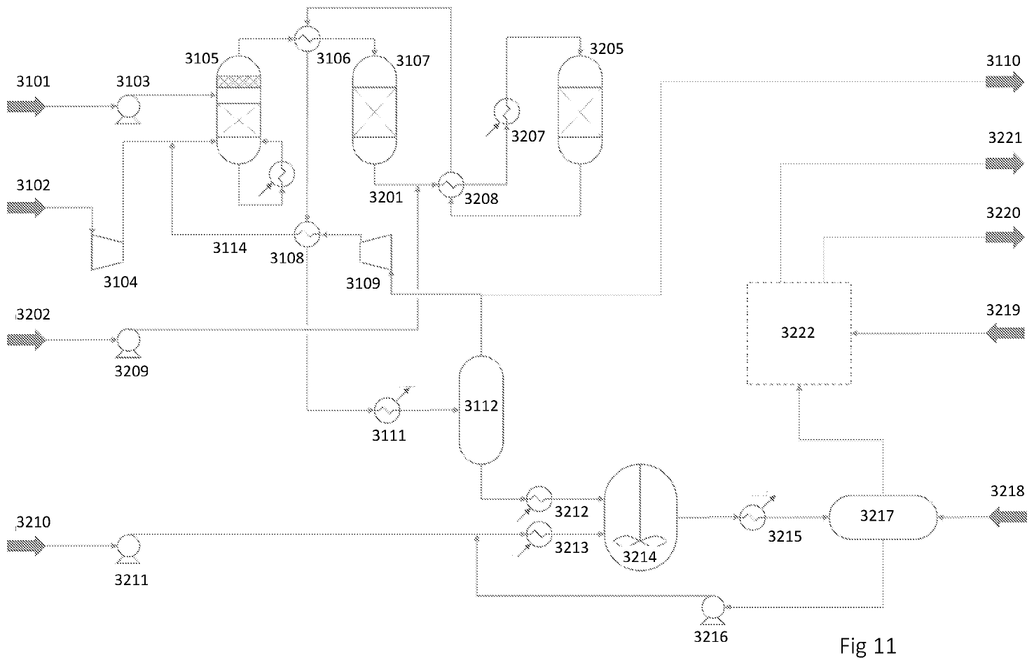

In FIG. 11, hydrogen gas 3102 is compressed 3104 and fed to a vaporiser 3105. Unsaturated heterocyclic amine, in this embodiment 3,5 lutidine 3101, is pumped 3103 into the vaporiser 3105, where it is vaporised into the hydrogen gas. The stream exiting the vaporiser comprises gaseous 3,5 lutidine and hydrogen gas and is heated 3106 and fed to a reactor 3107. In the reactor 3107 a vapour phase reaction between the 3,5 lutidine and the hydrogen takes place and 3,5 dimethylpiperidine is formed. The pressure in the reactor 3107 is preferably not more than 70 bar and the inlet temperature of the reactor 3107 is preferably in the range of from 150° C. to 350° C. Product 3201 from the reactor 3107 is mixed with methanol 3202 which is fed via pump 3209 and heated in heat exchanger 3208 and heater 3207 before being fed to N-alkylation reactor 3205. In the N-alkylation reactor 3205, the methanol forms dimethyl ether, which reacts with the 3,5 dimethylpiperidine, for example over an alumina catalyst, to produce 1,3,5 trimethylpiperidine. The N-alkylation reactor 3205 has an inlet temperature of at least 220° C. The product from the N-alkylation reactor 3205, which includes the 1,3,5 trimethylpiperidine is cooled in heat exchanger 3208, heat exchanger 3106, heat exchanger 3108 and cooler 3111, before being fed to a hydrogen knock-out pot 3112. In hydrogen knock out pot 3112, the product 1,3,5 trimethylpiperidine condenses and is produced as liquid 1,3,5 trimethylpiperidine, while the hydrogen remains as a gas and is produced overhead. The hydrogen is either compressed 3109 and recycled in hydrogen recycle stream 3114, or it is purged 3110. The liquid 1,3,5 trimethylpiperidine is heated 3212 and passed to second N-alkylation reactor 3214, to which is also fed dimethyl carbonate 3210 via pump 3211 and heater 3213. In the second N-alkylation reactor 3214 the 1,3,5 trimethylpiperidine may react with the dimethyl carbonate to form a mixture of 1,1,3,5 tetramethylpiperidinium carbonate, 1,1,3,5 tetramethylpiperidinium hydrogen carbonate and 1,1,3,5 tetramethylpiperidinium hydroxide. The product from the second N-alkylation reactor 3214 is cooled 3215 and fed to a dimethyl carbonate decanter 3217, to which water 3218 is also supplied. In the dimethyl carbonate decanter 3217, the dimethyl carbonate is separated and recycled via pump 3216, while the mixture of 1,1,3,5 tetramethylpiperidinium carbonate, 1,1,3,5 tetramethylpiperidinium hydrogen carbonate and 1,1,3,5 tetramethylpiperidinium hydroxide is sent to the counter-ion swap 3222.

There, the mixture of 1,1,3,5 tetramethylpiperidinium carbonate, 1,1,3,5 tetramethylpiperidinium hydrogen carbonate and 1,1,3,5 tetramethylpiperidinium hydroxide is contacted with sodium hydroxide or calcium hydroxide 3219, to convert the 1,1,3,5 tetramethylpiperidinium carbonate and 1,1,3,5 tetramethylpiperidinium hydrogen carbonate into 1,1,3,5 tetramethylpiperidinium hydroxide. A solution of sodium or calcium carbonate 3220 and a solution of the 1,1,3,5 tetramethylpiperidinium hydroxide 3221 are withdrawn from the counter-ion swap step 3222.

### Hydrogenation Examples

The process of FIG. 1 was tested in the following example run. Initially the reactor was charged with 115.8 g (equivalent to 150 ml) HTC 500RP, available from Johnson Matthey Plc, at a concentration of 0.772 g ml⁻¹.

The catalyst was activated by ramping the temperature to 230° C. from room temperature over 12 hours at 50 psig under a hydrogen flow of 50 normal l/h. The recycle compressor was in operation during the reduction, generating an approximate recycle flow of 300 g/h.

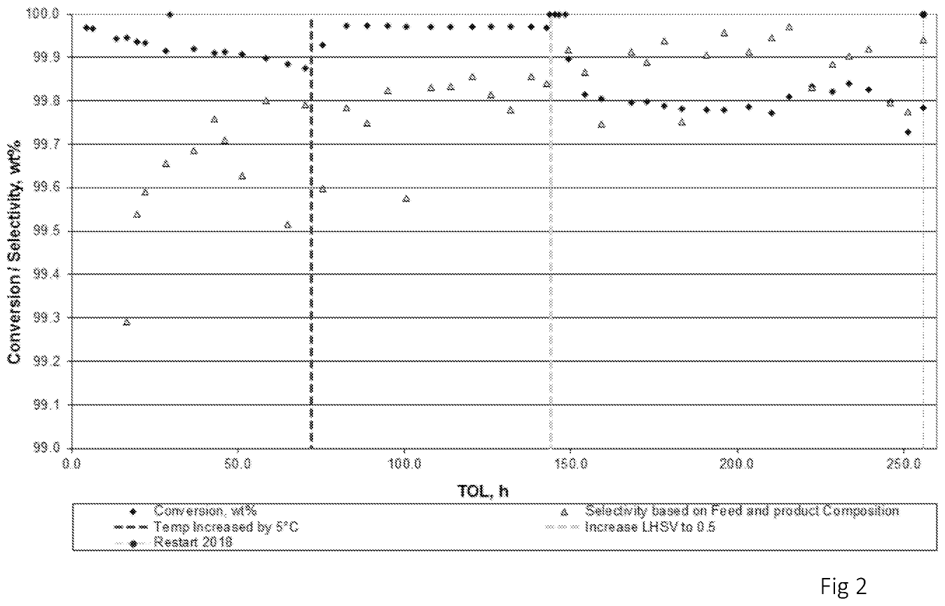

A feed of 3,5 lutidine was then introduced with a reactor inlet temperature of 185° C., a reactor pressure of 60 barg, a liquid hourly space velocity (LHSV) based on the liquid feed of 3,5 lutidine to the vaporiser divided by the volume of the catalyst of 0.4 h⁻¹ and a hydrogen: 3,5 lutidine ratio of 800 moles of hydrogen per mole of 3,5 lutidine. After approximately 40 hours on line the 3,5 lutidine conversion stabilised at approximately 99.91 wt %. After 70 hours on line the reactor inlet temperature was increased to 190° C. as the conversion had decreased to 99.88 wt %. This resulted in an increase in conversion to 99.97 wt %. After 143 hours on line the LHSV was increased to 0.5 h⁻¹. The conversion stabilised at approximately 99.78 wt %. These conditions were maintained until 256 hours on line. The lutidine conversion and catalyst selectivity for these 256 hours on line is shown in FIG. 2.

Following an extended shutdown, the unit was re-started at a reactor inlet temperature of 190° C., a reactor inlet temperature of 195° C., a reactor pressure of 60 barg, a LHSV of 0.5 h⁻¹ and a hydrogen: 3,5 lutidine ratio of 800 moles of hydrogen per mole of 3,5 lutidine.

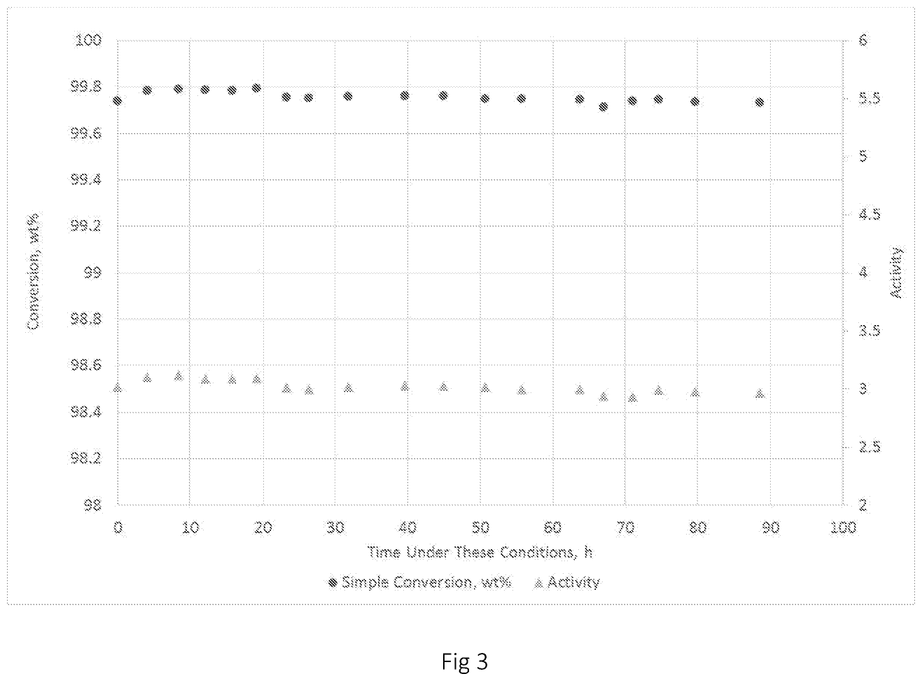

The conversion and activity for this next period of the run are shown in FIG. 3. It can be seen that there was no evidence of any significant loss of activity on re-start. This suggesting that the catalyst is stable enough to tolerate an enforced plant shutdown. The catalyst retained activity throughout the run, with a very slow deactivation rate.

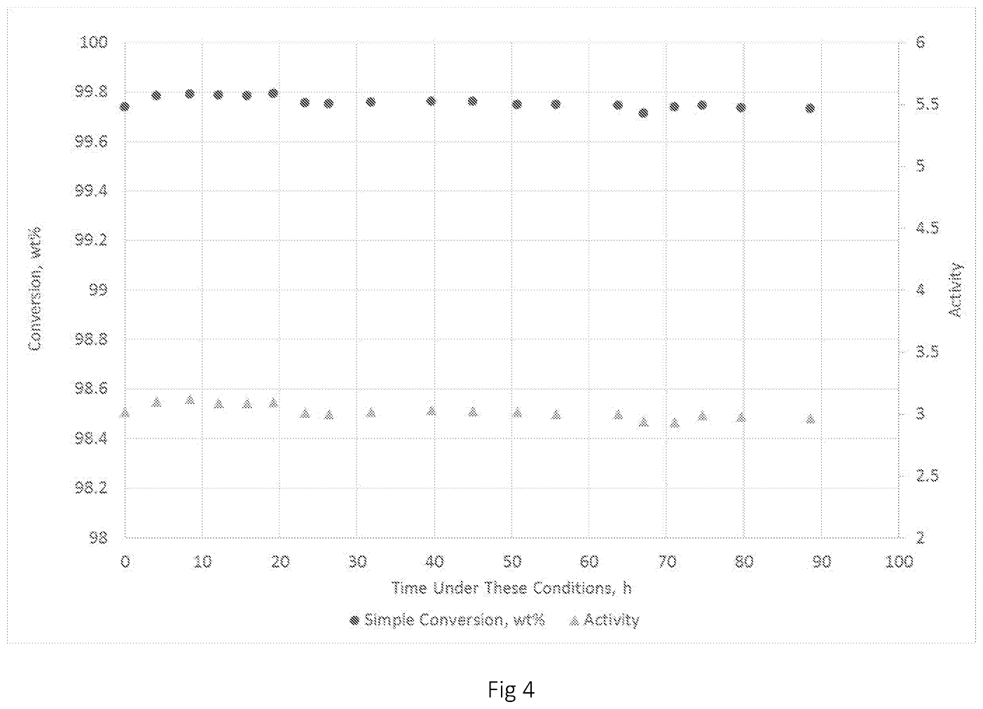

The recycle hydrogen gas was then reduced to produce a hydrogen: 3,5 lutidine ratio of 600 moles of hydrogen per mole of 3,5 lutidine, while all other parameters remained the same as before. The conversion and activity for this period are shown in FIG. 4, which demonstrates that there was no evidence of gross deactivation during this run.

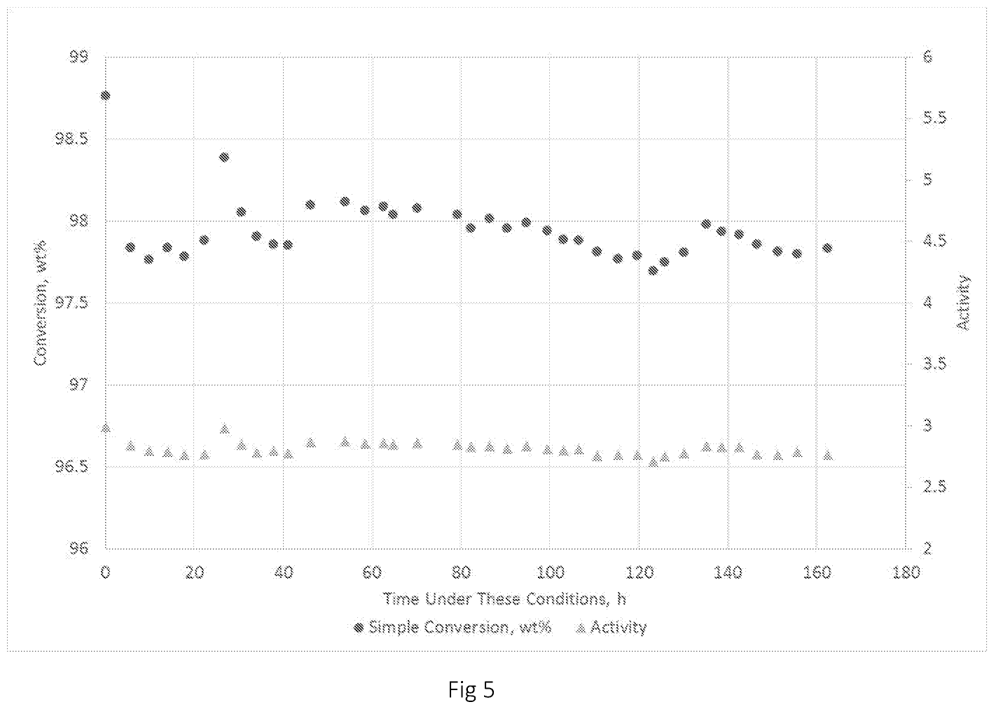

To achieve a hydrogen: 3,5 lutidine ratio of 400 moles of hydrogen per mole of 3,5 lutidine while maintaining a high gas flow the LHSV was increased to 0.72 h⁻¹ while keeping all other parameters the same as before. The conversion and activity for this period are shown in FIG. 5. There was no evidence of any significant increase in the rate of catalyst deactivation.

To simulate operation with an aged, less active catalyst, the LHSV was increased to 1.0, while maintaining a hydrogen: 3,5 lutidine ratio of 400 moles of hydrogen per mole of 3,5 lutidine.

After 660 hours on line this resulted in a 3,5 lutidine conversion of 92.76 wt %. There was no significant change in selectivity. For simplicity, assuming the drop-in conversion remains linear and starting at 99.5 wt % conversion then this is broadly equivalent to greater than 11000 hours on line, which is longer than the expected catalyst lifetime required for a commercial process.

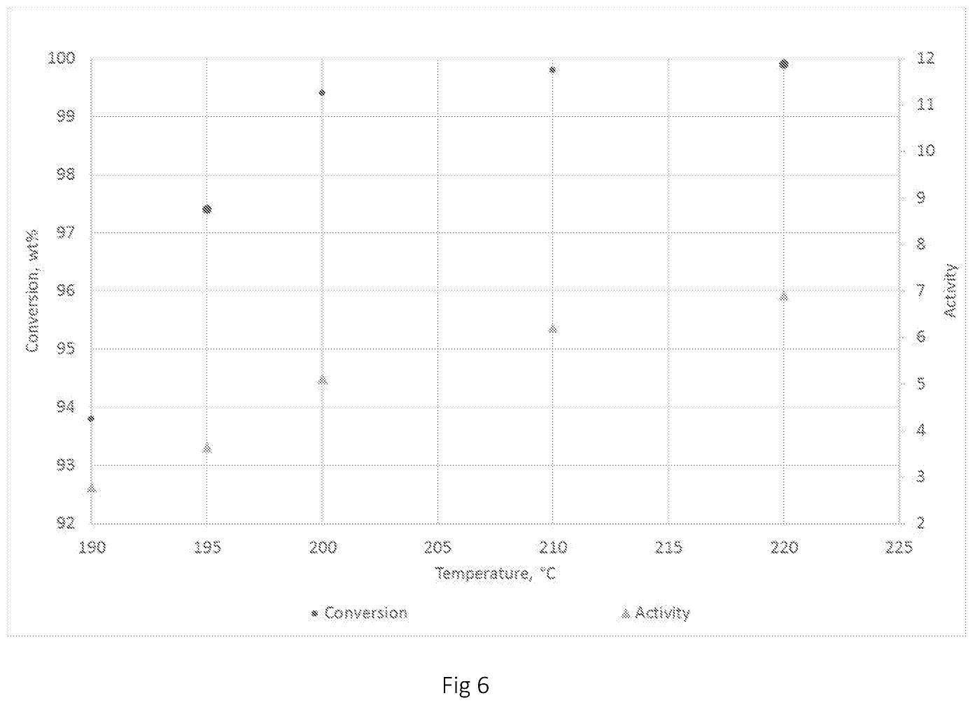

To simulate a catalyst management strategy whereby the catalyst temperature is increased to offset the loss of activity with time the temperature was increased over time, with no discernible change in product selectivity throughout. This is shown in Table 1 and FIG. 6

TABLE 1
Effect of Temperature on Conversion and Activity

| Run | 4 | 5 | 6 | 7 | 8 | 9 |
|---|---|---|---|---|---|---|
| Temperature, | 190 | 195 | 197 | 200 | 210 | 220 |
| ° C. | | | | | | |
| Time on | 660 | 708 | 731 | 773 | 794 | 804 |
| Line, h | | | | | | |
| Conversion, | 92.76 | 97.44 | 98.70 | 99.45 | 99.96 | 99.99 |
| wt % | | | | | | |
| Activity | 2.62 | 3.67 | 4.33 | 5.17 | 6.22 | 6.91 |

It can be seen that acceptable conversions and activity are obtained across the temperature range.

The unit was returned to at a reactor inlet temperature of 190° C., a reactor inlet temperature of 195° C., a reactor pressure of 60 barg, a LHSV of 0.5 h⁻¹ and a hydrogen: 3,5 lutidine ratio of 800 moles of hydrogen per mole of 3,5 lutidine. After 832 hours on line this resulted in a lutidine conversion of 99.58 wt % which equates to an activity of 2.80 suggesting that the catalyst has lost about 8% of activity over a period of at least 600 hours.

### First N-Alkylation Examples

### Vapour Phase N-Alkylation Example 1

An example vapour phase N-alkylation of 3,5 dimethylpiperidine to 1,3,5 trimethylpiperidine was carried out using a continuous vapour-phase process over a γ-alumina extrudate.

The reactor was operated at 190° C., with a pressure of 50 barg and a liquid hourly space velocity, based on the liquid feed rate of the 3,5 dimethylpiperidine prior to vaporisation divided by the volume of the catalyst, of 0.3 h⁻¹. The hydrogen gas rate through the reactor was 240 normal I/h. The feed to the reactor contained 10.14 wt % methanol, 7.3 wt % cis 3,5 dimethylpiperidine, 2.65 wt % trans 3,5 dimethylpiperidine and 79.8 wt % cyclohexane. The cyclohexane was present as an inert diluent to aid with pumping control and plays no part in the reaction. After 49 hours on line of continuous operation this resulted in a 3.4 wt % selectivity to 1,3,5 trimethylpiperidine.

Keeping all other parameters the same, the temperature was increased to 195° C. After 90.2 hours on line of continuous operation this resulted in a slightly higher selectivity to 1,3,5 trimethylpiperidine of 4.1 wt %.

As the conversion to 1,3,5 trimethyl piperidine was low, the temperature was increased to 225° C. After 115 hours on line of continuous operation this afforded a selectivity to 1,3,5 trimethylpiperidine of 14.8 wt %, which is a significant increase from the selectivity of 4.1 wt % at 195° C.

The temperature was further increased to 235° C. and the gas rate was reduced by 50% (from 240 to 120 normal I/h) to simulate an increased bed volume; noting that the liquid hourly space velocity was still the same as the previous run. After 137 hours on line of continuous operation this afforded a selectivity to 1,3,5 trimethylpiperidine of 32.9 wt %.

The temperature was increased to 250° C. and the gas rate reduced to 80 normal I/h. After 161 hours on line of continuous operation this resulted in an increased selectivity to 1,3,5 trimethylpiperidine of 57.1 wt %.

The gas rate was further reduced to 40 normal I/h. After 178.9 hours on line of continuous operation, this resulted in a selectivity to 1,3,5 trimethyl piperidine of 67.7 wt %.

The same reactor was then operated at a temperature of 250° C. a pressure of 50 barg, a hydrogen flowrate of 40 normal I/h, a LHSV of 0.23 h⁻¹ and a methanol to 3,5 dimethylpiperidine molar ratio of 8 moles methanol per mole of 3,5 dimethylpiperidine. After 195 hours on line this resulted in a conversion of about 80 wt % with 1,3,5 trimethylpiperidine isomers generated at high selectivity.

Keeping all other parameters the same, the temperature was increased to 275° C. After 223 hours on line this resulted in greater than 99 wt % conversion and good selectivity to 1,3,5 trimethylpiperidine isomers.

### Vapour Phase N-Alkylation Example 2

A further run was conducted, again using a continuous vapour-phase process over a γ-alumina extrudate. A feed of 29.78 wt % methanol and 68.66 wt % 3,5 dimethylpiperidine was fed to a reactor having an inlet temperature of 262° C., a reactor pressure of 10 barg, a liquid hourly space velocity based on the volumetric flowrate of liquid 3,5 dimethylpiperidine fed to the vaporiser of 0.79 h⁻¹. The methanol: 3,5 dimethylpiperidine molar ratio was thus 1.5:1. After 176 hours on line of continuous operation this afforded a 3,5 dimethylpiperidine conversion of 99.16 wt % with a product selectivity of 94.67 wt %.

Keeping all other parameters the same, the methanol: 3,5 dimethylpiperidine molar ratio was reduced to 1:1. After 212 hours on line of continuous operation this afforded a 3,5 dimethylpiperidine conversion of 96.28 wt % with a selectivity to the desired product of 92.82 wt %. The decrease in conversion is thought to be as a result of the absence of a molar excess of methanol to generate dimethyl ether. However, the conversion is still acceptable and this is thought to be because although 2 moles of methanol are needed to generate dimethyl ether, 1 mole of methanol is regenerated in the N-alkylation process and a 1:1 methanol: 3,5 dimethylpiperidine molar ratio therefore still gives an adequate result.

At 220 hours on line of continuous operation the feed was returned to a methanol: 3,5-dimethylpiperidine molar ratio of 1.5:1 and the LHSV increased to 1.0 h⁻¹. The reactor inlet temperature was reduced to 233° C. After 260 hours on line of continuous operation this resulted in a 3,5 dimethylpiperidine conversion of 99.33 wt % and selectivity of 97.39 wt %.

At 265 hours on line of continuous operation the LHSV was increased to 1.1 h⁻¹ with the other parameters remaining unchanged. After 308 hours on line of continuous operation this resulted in a 3,5-dimethylpiperidine conversion of 99.09 wt % and selectivity to 1,3,5-trimethylpiperidine of 94.26 wt %.

At 310 hours on line of continuous operation the LHSV was returned to 0.8 h⁻¹ with a methanol: 3,5-dimethylpiperidine molar ratio of 1.5:1 at a reactor inlet temperature of 261° C. These conditions were maintained for approximately 350 hours to generate catalyst deactivation data. After 683 hours on line of continuous operation this afforded a 3,5-dimethylpiperidine conversion of 99.15 wt % and a selectivity to 1,3,5-trimethylpiperidine of 92.50 wt %.

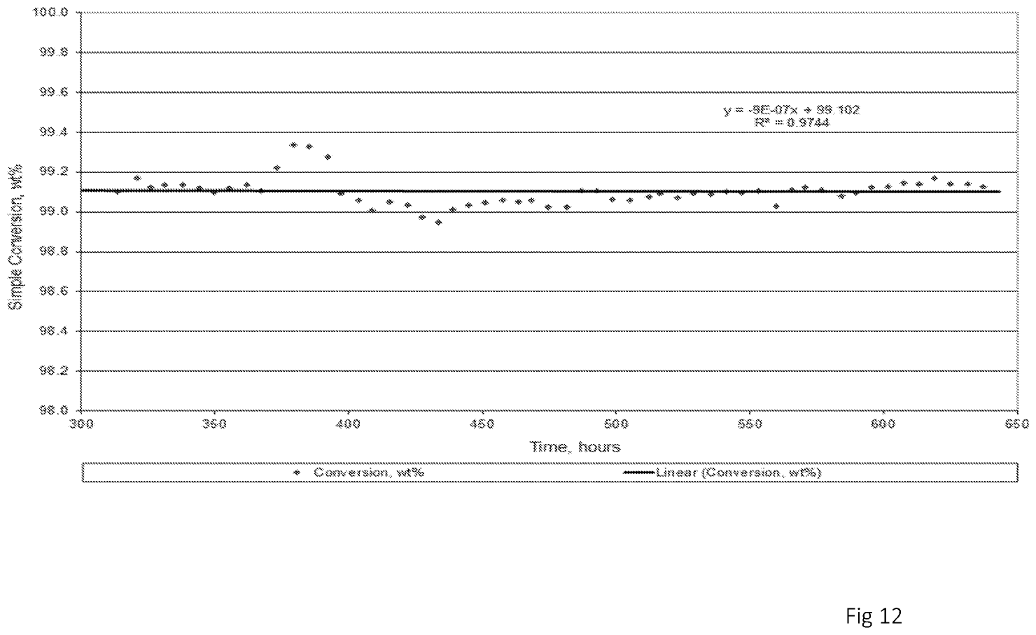

As shown in FIG. 12 the catalyst performance was very stable with only a slight decrease in activity seen.

### Vapour Phase N-Alkylation Example 3

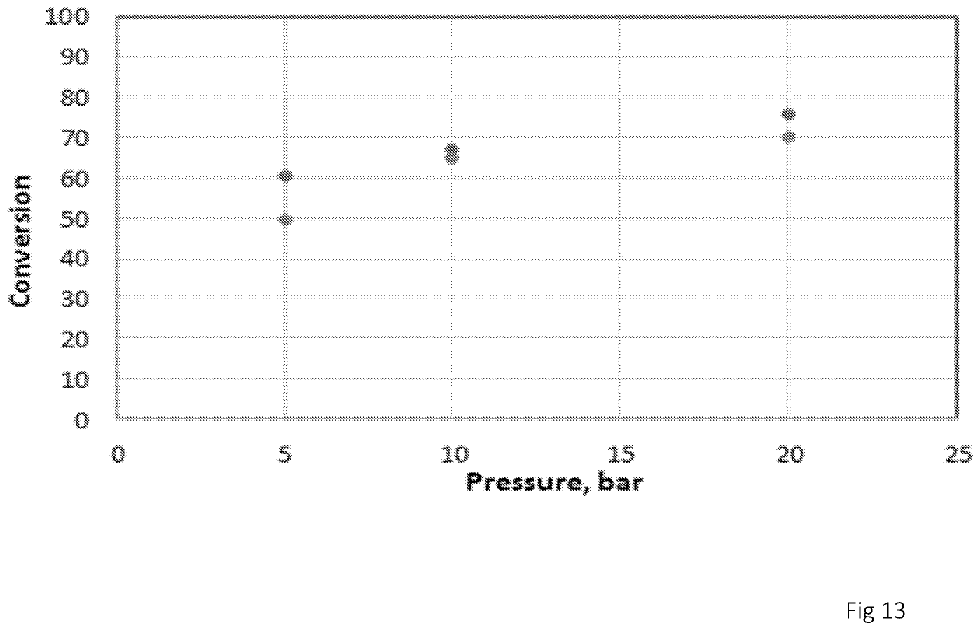

Three runs at a methanol: 3,5 dimethylpiperidine molar ratio of 2:1, an inlet temperature of 260° C., an LHSV of 2.2 h⁻¹ and pressures of 5 barg, 10 barg and 20 barg respectively were carried out in the continuous vapour phase reactor. These three runs show the impact of pressure on the process. For each run, two different silica/alumina catalysts having 3% silica were used. Both catalysts showed adequate conversion. The results for conversion in wt %, which are shown in FIG. 13, indicate that by increasing the pressure the 3,5 dimethylpiperidine conversion increases, although adequate conversion is observed at all pressures.

### Vapour Phase N-Alkylation Example 4

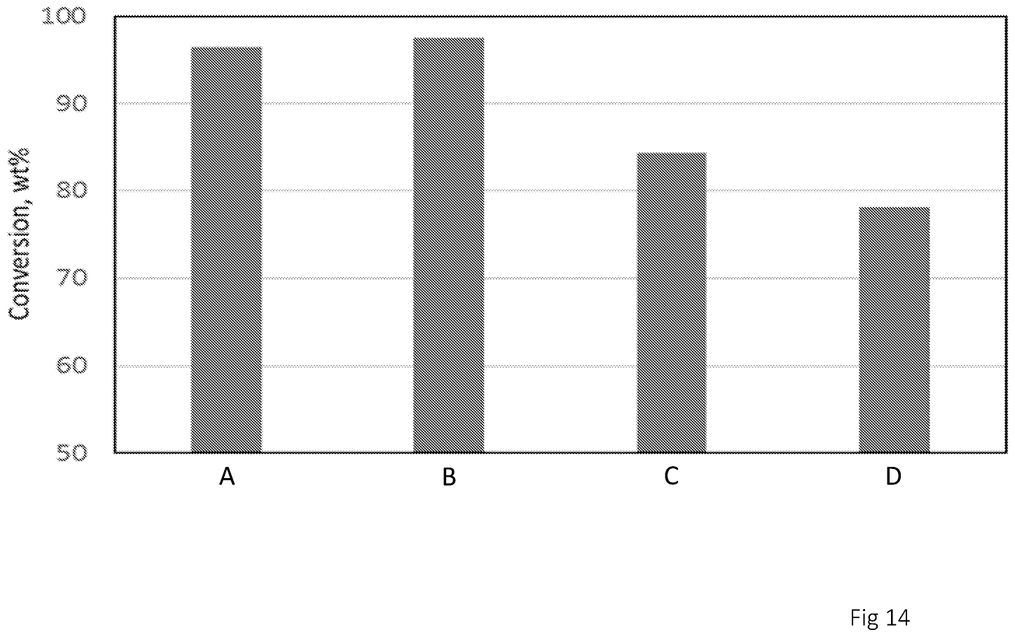

A further experiment was carried out to compare 4 different catalysts. Catalysts A and B are silica/alumina catalysts having 3% silica, while catalysts C and D are alumina catalysts. The catalysts were used in a continuous vapour phase N-alkylation process at a methanol: 3,5 dimethylpiperidine molar ratio of 5:1, an inlet temperature of 260° C., an LHSV of 0.7 h⁻¹ and a pressure of 10 barg. The results in FIG. 14 show that the average conversion for both types of catalyst was acceptable, but that the silica/alumina catalysts A and B had higher average conversions.

### Vapour Phase Hydrogenation and N-Alkylation

A combined vapour phase hydrogenation and first N-alkylation may be achieved by the addition of methanol to the feed to the hydrogenation reactor to promote the gas phase N-alkylation reaction co-current with the hydrogenation reaction, via a dimethyl ether intermediate. This may be advantageous in reducing the number of reactors required. However, the formation of methane and water would potentially require a large purge from the hydrogen gas recycle to maintain an appropriate recycle gas molecular weight.

A feed composition of 9.6 wt % 3,5 lutidine, 10.1 wt % methanol and the remainder cyclohexane was vaporised and fed to a reactor operated at a temperature of 190° C., a pressure of 50 barg, an LHSV based on the volumetric liquid flow rate of 3,5 lutidine to the vaporiser divided by the volume of catalyst in the reactor of 0.3 h⁻¹, a gas rate through the reactor of 240 normal l/h and a hydrogen: 3,5 lutidine ratio of 800 moles of hydrogen per mole of 3,5 lutidine. The cyclohexane is an inert diluent to allow for pumping control and plays no part in the reaction.

After 360 hours on line of continuous operation, this resulted in a 3,5 lutidine conversion of 98.77 wt %, with 31.7 wt % selectivity to 1,3,5 trimethylpiperidine, 50.0 wt % selectivity to cis 3,5 dimethylpiperidine and 18.3 wt % selectivity to trans 3,5 dimethylpiperidine.

Keeping all other parameters the same, the feed was changed to 10.1 wt % 3,5 lutidine and 89.8 wt % methanol. After 396 hours on line of continuous operation, this resulted in a 3,5 lutidine conversion of 99.23 wt % with 93.5 wt % selectivity to 1,3,5 trimethylpiperidine, 4.65 wt % selectivity to cis 3,5 dimethylpiperidine and 1.85 wt % selectivity to trans 3,5 dimethylpiperidine. The unit was operated at these conditions until 538 hours on line of continuous operation, at which point the 3,5 lutidine conversion was 99.80 wt % suggesting no loss of activity over this period of operation.

### Comparative Example of Batch N-Alkylation

20 ml 3,5 dimethylpiperidine was transferred into 200 g hot dimethyl carbonate at 200° C. in a 300 ml autoclave via a pump. Transferring the 3,5 dimethylpiperidine into the already hot dimethyl carbonate avoided the formation of unwanted carbamate when heating the autoclave. Samples were taken from a submerged dip-leg and analysed by gas chromatography (GC). The results are in table 2 below.

TABLE 2
Batch Liquid-Phase First N-Alkylation

| | GC wt % | | | | | | | | | |
|---|---|---|---|---|---|---|---|---|---|---|
| | | | 1,3,5- | Cis-3,5 | Trans-3,5 | | | | 3,5 dimethyl- | 1,3,5 trimethyl- |
| Time | | Dimethyl | trimethyl- | dimethyl- | dimethyl- | 3,5 | | | piperidine | piperidine |
| (mins) | Methanol | carbonate | piperidine | piperidine | piperidine | Lutidine | Carbamate | Total | conversion wt % | selectivity wt % |
| 0 | 0.34 | 99.29 | 0.04 | 0.18 | 0.03 | 0.01 | 0.06 | 99.61 | | |
| 1 | 0.81 | 33.95 | 1.12 | 45.77 | 15.85 | 0.13 | 1.66 | 98.48 | | |
| 5 | 2.29 | 81.22 | 4.25 | 6.82 | 2.48 | 0.03 | 2.72 | 97.52 | | |
| 10 | 2.38 | 90.18 | 3.33 | 0.67 | 0.26 | 0.08 | 2.84 | 97.36 | 89.97 | 46.88 |
| 15 | 2.37 | 90.25 | 3.47 | 0.66 | 0.22 | 0.06 | 2.70 | 97.36 | 90.47 | 49.19 |
| 30 | 2.53 | 89.86 | 3.50 | 0.71 | 0.24 | 0.03 | 2.84 | 97.19 | 89.80 | 48.03 |
| 60 | 2.81 | 90.21 | 3.63 | 0.12 | 0.01 | 0.05 | 2.71 | 96.74 | 98.62 | 56.11 |
| 120 | 3.28 | 90.05 | 3.36 | 0.06 | 0.00 | 0.06 | 2.55 | 96.09 | 99.29 | 56.21 |

The samples are not representative of the autoclave contents until the 10-minute sample, because the 3,5 dimethylpiperidine was fed into the autoclave via the same dip-leg that was used to take the samples. The 3,5 dimethylpiperidine conversion reached 99.3 wt % after 120 minutes. However, the selectivity to the target 1,3,5 trimethylpiperidine was only 56.2 wt %, with significant losses to the carbamate species. There was also a quantity of solid precipitate produced during the test. This is likely the 1,1,3,5-tetramethylpiperidinium methyl carbonate salt that is formed by the second methylation step. This precipitate accounted for as much as 10 wt % of the samples taken.

Compared to the vapour phase N-alkylation example above, which affords a high conversion of 3,5 dimethylpiperidine and a high selectivity to 1,3,5 trimethylpiperidine, the batch autoclave N-alkylation was not very selective to the target 1,3,5 trimethylpiperidine and formed significant quantities of unwanted carbamate.

It will be appreciated by persons skilled in the art that the above embodiments have been described by way of example only, and not in any limitative sense, and that various alterations and modifications are possible without departure from the scope of the invention as defined by the appended claims. For example, while the above examples use 3,5 lutidine as a feed to the hydrogenation, other unsaturated heterocyclic amines are contemplated as part of the invention. As explained above, the same deactivation issues that occur with 3,5 lutidine hydrogenation in the liquid phase would be expected to occur with other unsaturated heterocyclic amines and would be expected to be solved in the same manner by the process of the invention. Similarly, the advantages demonstrated above in relation to vapour phase N-alkylation of 3,5 dimethylpiperidine would be expected to apply to N-alkylation of other saturated heterocyclic amines.

1. A process of N-alkylating a saturated heterocyclic amine, the process comprising N-alkylating the saturated heterocyclic amine in a vapour phase reaction at a temperature of at least 220° C.

2. A process according to claim 1 wherein the process comprises reacting the saturated heterocyclic amine with dimethyl ether.

3. A process according to claim 2, wherein the dimethyl ether is generated in situ by the etherification of methanol.

4. A process according claim 3, wherein the saturated heterocyclic amine and the method are fed to the process at a molar ratio of from at least 1 mole of alkanol per mole of saturated heterocyclic amine to not more than 20 moles of alkanol per mole of saturated heterocyclic amine.

5. A process according claim 1, wherein the pressure is in the range of from 1 bar to 100 bar.

6. A process according to claim 1 wherein the gas hourly space velocity is in the range of from 20 h⁻¹ to 200 h⁻¹.

7. A process according to claim 1, wherein the saturated heterocyclic amine comprises a piperidine ring or a pryyolidine ring.

8. A process according to claim 7, wherein the saturated heterocyclic amine is selected from the group consisting of: piperidine, 2-methyl piperidine, 3-methyl piperidine, 4-methyl piperidine, 2,6 dimethylpiperidine, 3,5 dimethylpiperidine, pyrrolidine, 2-methyl pyrrolidine, 3-methyl pyrrolidine, 2,4-dimethyl pyrrolidine, and 2,5-dimethyl pyrrolidine.

9. A process according to claim 1, wherein the saturated heterocyclic amine is a fully saturated heterocyclic amine.

10. A process according to claim 1, wherein the saturated heterocyclic amine is 3,5 dimethylpiperidine and the process produces an N-alkylation product comprising 1,3,5 trimethylpiperidine.

11. A process according to claim 1 wherein the process comprises feeding gaseous saturated heterocyclic amine to a reactor, N-alkylating the saturated heterocyclic amine in a vapour phase reaction in the reactor to produce an N-alkylation product, wherein the reactor inlet temperature is at least 220° C., and withdrawing a product stream comprising the N-alkylation product from the reactor, wherein the feeding and withdrawing are continuous.

12. A process according to claim 11, wherein liquid saturated heterocyclic amine is fed to a vaporiser where it is vaporised along with an alkylating reagent to create a mixed gaseous stream of alkylating reagent and saturated heterocyclic amine that is then fed to the reactor.

13. A process according to claim 12, wherein the alkylating reagent is methanol.

14. A process according to claim 1, wherein the vapour phase reaction occurs over a catalyst comprising alumina.

15. A process according to claim 14, wherein the catalyst comprises λ-alumina.

16. A process according to claim 14, wherein the the catalyst also comprises silica.

17. A process for the production of heterocyclic quaternary ammonium salts or hydroxides, the process comprising a continuous hydrogenation step, in which an unsaturated heterocyclic amine is reacted with hydrogen to form a saturated heterocyclic amine; a first continuous N-alkylation step, in which the saturated heterocyclic amine is N-alkylated to produce an intermediate saturated heterocyclic amine having an increased degree of substitution compared to the saturated heterocyclic amine; and one or more further N-alkylation steps in which the intermediate saturated heterocyclic amine is N-alkylated to the heterocyclic quaternary ammonium salt or hydroxide.

18. A process according to claim 17, wherein the the process further comprises a counter ion swap step, in which a first counter ion on the heterocyclic quaternary ammonium salt or hydroxide is exchanged for a second counter ion.

19. A process according to claim 18, wherein the counter ion swap step is continuous.

20. A process according to claim 17, wherein the the hydrogenation step is in the vapour phase.

21. A process according to claim 17 13, wherein the the first continuous N-alkylation step is in the vapour phase.

22. A process according to claim 17, wherein the one or more further N-alkylation steps are continuous.

23. A process according to claim 17, wherein the unsaturated heterocyclic amine comprises a pyridine ring or a pyrrole ring, the saturated heterocyclic amine comprises a piperidine ring or a pyrrolidine ring, the intermediate saturated heterocyclic amine comprises a piperidine ring or a pyrrolidine ring, and the heterocyclic quaternary ammonium salt or hydroxide comprises a piperidinium ring or a pyrrolidinium ring.

24. A process according to claim 23, wherein the unsaturated heterocyclic amine is selected from the group consisting of: pyridine, 2-methyl pyridine, 3-methyl pyridine, 4-methyl pyridine, 2,6 lutidine, 3,5 lutidine, pyrrole, 2-methyl pyrrole, 3-methyl pyrrole, 2,4-dimethylpyrrole, and 2,5-dimethylpyrrole; the saturated heterocyclic amine is selected from the group consisting of: piperidine, 2-methyl piperidine, 3-methyl piperidine, 4-methyl piperidine, 2,6 dimethylpiperidine, 3,5 dimethylpiperidine, pyrrolidine, 2-methyl pyrrolidine, 3-methyl pyrrolidine, 2,4-dimethyl pyrrolidine, and 2,5-dimethyl pyrrolidine; the intermediate saturated heterocyclic amine is selected from the group consisting of: 1-methylpiperidine, 1,2-dimethyl piperidine, 1,3-dimethyl piperidine, 1,4-dimethyl piperidine, 1,2,6 trimethylpiperidine, 1,3,5 trimethylpiperidine, 1-methylpyrrolidine, 1,2-dimethyl pyrrolidine, 1,3-dimethyl pyrrolidine, 1,2,4-trimethyl pyrrolidine, and 1,2,5-trimethyl pyrrolidine; and the heterocyclic quaternary ammonium salt or hydroxide is selected from the group consisting of: 1,1-dimethylpiperidinium salt or hydroxide, 1,1,2-trimethylpiperidinium salt or hydroxide, 1,1,3-trimethylpiperidinium salt or hydroxide, 1,1,4-trimethylpiperidinium salt or hydroxide, 1,1,2,6 tetramethylpiperidinium salt or hydroxide, 1,1,3,5 tetramethylpiperidinium salt or hydroxide, 1,1-dimethylpyrrolidinium salt or hydroxide, 1,1,2-trimethylpyrrolidinium salt or hydroxide, 1,1,3-trimethylpyrrolidinium salt or hydroxide, 1,1,2,4-tetramethyl pyrrolidine salt or hydroxide, and 1,1,2,5-tetramethyl pyrrolidine salt or hydroxide.

25. A process according to claim 24, wherein the unsaturated heterocyclic amine is 3,5 lutidine, the saturated heterocyclic amine is 3,5 dimethylpiperidine, the intermediate saturated heterocyclic amine is 1,3,5 trimethylpiperidine and the heterocyclic quaternary ammonium salt or hydroxide is 1,1,3,5 tetramethylpiperidinium hydroxide.

26. A process according to claim 17, wherein the hydrogenation step comprises reacting an unsaturated heterocyclic amine with hydrogen in a vapour phase reaction at a pressure of not more than 70 bar and a temperature in the range of from 150° C. to 350° C.

27. A process according to claim 17, wherein the first N-alkylation step comprises N-alkylating the saturated heterocyclic amine in a vapour phase reaction at a temperature of at least 220° C.

28. A process according to claim 17, wherein the wherein the saturated heterocyclic amine is collected from the hydrogenation step as a condensed liquid and vaporised and fed to the first N-alkylation step.

29. A process according to claim 28 wherein the collection is carried out continuously.

30. A process according to claim 29, wherein the collection is carried out by passing a product stream from the hydrogenation step to a knock-out pot in which the saturated heterocyclic amine is condensed and separated from hydrogen in the product stream.

31. A process according to claim 17, wherein a product stream from the hydrogenation step, comprising the saturated heterocyclic amine and hydrogen, is fed directly to the first N-alkylation step.

32. A process according to claim 17, wherein the product stream is a gas stream and the product stream is kept in the gas phase and fed to the first N-alkylation step.

33. A process according to claim 17, wherein the hydrogenation step and the first N-alkylation step are carried out in the same reactor.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)
